SEQUENCE LISTING (57 sequences)

SEQ ID NO: 1 - protein, 1046 aa, Streptococcus pyogenes
MTNCKYKLRKLSVGLVSVGTMLIAPTVLVQEVSASTTSTETSTASAGVGTSGTAASETGSGAAVTTATTTTATTNGGPQSTPAVAEATPQPQAQIAPVAAATSTSSASSSSDGKAPQAVTSSTSPSTPAAASSNGSNQEASAETEPQTMEVEKYTVDKENSKLNIKDGKTPKTGSSVNNEKDTKLIRNRDGKLRDIVDVTRTVKTNEDGTIDVTVTVKPKQIDEGADVMALLDVSKKMSEDDFNNAKNKIKKLVKTLTSKSASNSDNDEHKYNSRNSVRLMTFYREISNPIDISGKTEEQLDKLLDDLRKKAKANYDWGVDLQGAIHKAREIFNKEKEKKFGKRRHIVLFSQGESTFSYELQNSVREDKTKLSRLSGAVTSSNPLLPWPPIFNHTHKNIDMLDDVKNLVKLGQTLGIAGLDNLQSTLSLISTGSSLAGAFLGGGSLTEYLTLKEYKSGDLKENQFDYTKRVGEGYHFHSFSERKKTGEIPFKSEIEPKIKELFENNKNNQDKSWTEWIFDKLSLTERIQKAKQETLMKLLEYLFYKREYHYYNHNLSAIAEAKMAQQEGITFYSVDVTDLKTTSKRVKRQVESTEDKKKEKDREDIEKERNEKFDNYLKQMSEGGKDFFEDVDKAEKFKDILTNVTVTETFEDGVNVKDNSWQVSSENNNSLHSNYKSVTHKAASDASWWSLYSNKESLTWTISKEQLKEAFEKNSSLTFKYKLQVNKQKLLDKNKNRTKRDTSTENKTSVTKDIISNTVNYKINNQEVKGNKLDDVKLTYTKETVPVPDVEGEVVPIPEKPLVEPMTPLYPAIPNYPTPDIPTPQLPKDEDLEISGGHGPSVDIVEDTGTGAEGGAQNGVVSTQENRDPIVDITEDTQPGMSGSNDATVVEEDTAPKRPDVLVGGQSDPIDITEDTQPSVSGSNDATVVEEDTVPKRPDSLVGGQSDPIDITEDTQPGMSGSNGATVIEEDTRPKRVFHFDNEPQAPEKPNEQPSLSLPQAPVYKAAHHLPASGDKREASFTIAAPTIIGAAGLLSKKRRDTEGN

SEQ ID NO: 2 - DNA, 3141 bp, Streptococcus pyogenes
atgacaaattgtaagtataaacttagaaagttatctgtagggctcgtctccgtcggaacgatgctgatagccccgacagttttagttcaggaggttagtgctagtactactagtactgagacgagtactgctagcgctggtgtcggtacgagtgggacggccgccagcgaaactgggagtggagcagccgtaactactgccactactaccaccgctactaccaatggaggaccccagtctactccagcagtagctgaagcgactccacaacctcaagcacagatagctccagtagcagcagcaacgtcgacatcatcggcttcttctagtagtgacgggaaagctcctcaggcagtaacttcatctacatcaccttcaactccagcagcagccagtagtaatggtagcaatcaagaagctagtgctgagactgagccacagacgatggaagtggaaaagtatacagttgataaggaaaattcaaagctaaatattaaagacggtaagactccaaaaactgggagtagtgttaataatgaaaaagacacaaaacttattagaaaccgcgatggcaaacttcgtgatattgttgatgttactcggacagttaaaactaacgaagatggcactattgatgttaccgtaacggttaaaccgaagcaaattgacgaaggtgccgatgttatggcccttttagatgtctctaaaaagatgtcagaagatgattttaacaacgctaagaataagatcaagaaattagtcaaaaccttaacgagtaaatcagcgagtaactcagataatgatgagcataaatataattctcgaaattcggttcgtctgatgaccttttaccgtgagattagcaacccaattgatatatcaggaaaaaccgaggaacaacttgataaattattagacgatcttcgcaaaaaagctaaagctaattatgactggggggttgatttacagggagctatccacaaggctcgagagatttttaataaggaaaaagaaaaaaaatttggtaaacgccggcatatcgtcctattctctcaaggcgagtcaacctttagttatgaacttcaaaatagtgttagagaagataaaactaagttatcccgattaagtggagcagttacttcgtccaaccctctgctaccctggccacctatttttaatcatacgcataaaaatatagacatgcttgacgatgtaaagaatttggtaaaactaggtcaaactttaggaattgcagggctagataatttacagagtacattgagcttaatatcgacaggaagttctctggcaggagcgtttttagggggggggagtctgacagaatacctcactctaaaggagtataaatcaggagacttaaaagaaaatcagtttgattataccaaacgtgttggtgaaggatatcatttccatagtttttctgagagaaaaaaaactggcgaaataccgtttaagagtgaaatagaaccaaaaataaaagaattatttgaaaataacaagaataatcaagataaatcatggactgagtggatatttgataaattatcactgacagagagaattcaaaaagctaagcaggaaacacttatgaagctgcttgaatacctcttttacaaacgtgaataccactactataatcacaacctctcagcgatagctgaagctaaaatggctcaacaagaaggtatcaccttctattccgttgatgttactgatttaaaaacaacttctaaaagagtgaagcgacaagtagaaagtacagaggataagaaaaaagaaaaagatagggaagacattgaaaaagaacgtaacgaaaagtttgataattacttaaaacaaatgtctgaaggcggtaaggatttttttgaagatgttgataaggcagaaaaatttaaagatatcttaactaatgtaacggtgaccgagacttttgaagatggggttaacgttaaggataattcatggcaagtttcatcagagaataataatagcttacatagtaattataagagtgttacacataaagcagcatctgatgcaagttggtggtctttgtatagtaacaaagaaagtcttacttggaccatttctaaagagcagctcaaagaagcctttgagaaaaatagttctctcactttcaagtacaagttacaggtaaataaacaaaaactattagataaaaacaagaatagaacaaaacgtgatacatctacggaaaataagacttctgtaacgaaagacattatttcaaatactgttaactacaaaattaataatcaagaagttaagggtaacaaacttgatgatgtcaagttgacttatactaaagagaccgttcctgttccagatgtggaaggagaagttgtaccaataccagaaaaaccactggtagagccaatgacgcctctatatcctgcaattcctaattacccaacaccagatatccctacccctcaacttccaaaagatgaagatctggagattagtggaggtcatggaccgagtgtcgatatcgtcgaagatactggtacaggtgctgagggcggcgctcaaaacggcgtggtttcaactcaggagaatagagatccaatcgttgacatcaccgaagatacccaaccaggtatgtcaggctcaaatgacgcgacagttgtcgaggaagacacagcacctaaacgtccagatgtccttgttggtggtcaaagtgatccaatcgatatcaccgaagatacccaaccaagtgtgtcaggctcaaatgacgcgacagttgtcgaggaagacacagtacctaaacgtccagatagccttgttggcggtcaaagtgatccaatcgacatcaccgaagatacccaaccaggcatgtcaggctcaaatggcgctactgttatcgaagaagatacgagaccaaaacgcgtcttccactttgataacgagccacaagcaccagaaaaacctaacgagcaaccatctctcagcttaccacaagcgccagtctataaggcagctcatcacttgcctgcatctggagacaaacgtgaagcatcctttacaattgctgctccaacaattattggagctgcaggtttgctcagcaaaaaacgtcgcgacaccgaaggaaactaa

SEQ ID NO: 3 - protein, 954 aa, Streptococcus pyogenes
  VARIANT (1)...(954): Xaa = Any Amino Acid
SSTKTSASTNINTNTSTASAGTGTSGTASTTPSVGTSTGGAAGGEAAVASSGGSQSSESAQASTQPQAQTAVAASASTTASPSSSEEKTPKTVTSSTSSTPAASSSSNGNQVTGTEVEPQMMDVEQYKVNKEKTELTVKDDKQQLKIRKDVELKNKDPFDVKREVKDNGDGTLDVTLKVMLKQIDEGADVMALLDVSQKMTQENFNKAKEQIKRLVTTLTGKSSDGKENHNRRNSVRLMTFYRKISEPIDLSGKTSDEVEKELNKIWDKVKKEDWDWGVDLQGAIHKAREIFRSSYEKKSGKRQHIVLFSQGEPTFSYDIKNKNDNKLTKARIEEEVTSSNPLLSWPPIFNHTNRKADMLNDIEYLIKLGERLGITGLDSLKNTLKLASTGSSIAGSLLGSGSLSEYLTLKEYESRTLKESNFDYTKRVGEGYYYHSFSERIQNELPLKSIIEPQLKGLFKTEDSSWFGRFLNKFSLAKGYLRIKETALLKVLEYLFYKREYIYYNHNLSAIAEAKMAQQEGITFYSVDVTSPNQSANKRTRRSADTPEEKRNKKFDNYLKECPEGRKFFRRVRMSQIKDKFKDTLTELTIKDEFXEKVTVHKDKEYYKTSLIDDRPKVTHQAPYSSWLNSTKESLTWTISKDQLKKAFESGQPLTLTYKLKVEKEKFKEALKKQQERKKRAASPESENTVTDTIISNKISYKINNGTDINSNNNKLEDVKMSYSKFKMPIPELDIEVVPIPEKPLVEPMTPLYPAIPNYPTPDIPTLQLPKDEDLEISGGHGPIVDIVEDTGTGVEGGAQTGVVSTQENKDPIVDITEDTQPGMSGSNDATVVEEDTTPKRPDVLVGGQSDPIDITEDTQPSVSGSNDATVIEEDTKPKRFFHFDNEPQAPEKPKEQPSLQDSNSLPQAPAYKAAHHLPASGDKREVYFTIAALTIIGAAGLLSKKRRDTEEN

SEQ ID NO: 4 - DNA, 803 bp, Streptococcus pyogenes
  misc_feature (1)...(803): n = A,T,C or G
gaagttttagaaangaangccnctaaanacccnaggaaagctttngaattaaannaaaggtngtnacccnaataaccccaagaaaaaaaccccacttgggtanaaacccaaatgagcgcccttttnnntccctggcaatttcctaattnancccaaacaccaggatatccctaccnttcaacttcccaaaaagatgaagancctggagattagtggaggtcatggacccgattgtcgatatcgtcgaagatactggtacaggtgttgagggcggcgctcaaaacggcgtggtttcaactcaggaaaataaagatccaatcgttgacatcacggaagatacccaaccaggtatgtcaggctctaatgacgcaacagttgtcgaggaagacacaacacctaaacgtccagatgttcttgttggtggtcaaagtgatccaatcgacatcactgaagacacccaaccaagtgtatcaggctctaatgacgctactgttatcgaagaagatacgaaaccaaaacgcttcttccactttgataacgagccacaagcaccagaaaaacctaaagagcaaccatctcnacaagatagtaacagcttaccacaagctccagcctataaggcagctcatcacttgcctgcatctggagacaaacgtgaagtatactttacaattgctgctctaacaattattggagctgcaggtttgctcagcaaaaaacgtcgcgacnccgaagaaaactaaaagcttcccaagggcgaattccagcacactngcggccgttactagtggatccgagctngg

SEQ ID NO: 5 - protein, 1026 aa, Streptococcus pyogenes
MTNCKYKLRKLSVGLVSVGTMLIAPTVLGQEVSASSSTESSTTTANTGTGTASGMTATTPSATTDTGEAAGSGARSEANGASSVVSSEESQSSGTTPASPQAQTAPAATSTSSVSSSNEKTPKTATTTTSSTPVASTSNNSNKVTSTEAETQTVDVERYTVDKENSKLNIKDGKTPKTRSSVNKDTKLIRNRDDKQRDIVDVTRTVETNEDGLLMFTGNGLKPKQIDEGADVMALLDVSQKMTKENFDKAKEQIKKMVTTLTGEPTDGKENHNRRNSVRLMTFYRKVNEPIELTAENVDKTLDEVWKKAKEDWDWGVDLQGAIHKAREIFNKEKEKKSGKRQHIVLFSQGESTFSYDIKNKMILQKLPITEKVTTSSPLFPWLPIFNHTNRKAEIIGDLEKVLDMAEKVGISLPSSLKSAVKVLGLTNSAIGSILGKGLTEYLGLTEYSSDNLDGGGFDYSKRVGEGYYYHSLSDRKYENTMPLEEAIRTALASNFPKLTDNWFFDILNSFVNKDTVEKAKLDVIMKVLNSIFYKKEYRYYNHNLSAIAEAKMAQQEGITFYSVDVTDLNSASKRVRRQAAVRKGTKEENKKNEEERNTKFDTYLKKMSEGNNFLSNVEERDFFKDTLTELTIKDEFTDKVTVEKDSWSKSITDGLKNSNNNNVKHQQANTSTWSFFSSSKESLTWIISKEALKETFEKNGSLTFKYKLRVNKDKLLDLDKKETKRDTSTENKTSVTANIISNTVNYKINNQEVKGNKLDAVNLTYTKETVPVPDVEGEVVPIPEKPLVEPMTPLYPAIPNYPTPQLPKDEDLEISGGHGPIVDIVEDTGAGVEGGAQNGVVSTQENKDPIVDITEDTQPGMSGSNDATVVEEDTAPKRPDVLVGGQSDPIDITEDTQPGMSGSNDATVIEEDTAPKRPDVLVGGQCDPIDITEDTQPGMSGSNDATVIEDDTKPKRFFHFDNEPQAPEKPKEQPSLSLPQAPVYKAAHHLPASGDKREASFTIVALTIIGAAGLLSKKRRDTEEN

SEQ ID NO: 6 - DNA, 3081 bp, Streptococcus pyogenes
atgacaaattgtaagtataaacttagaaagttatctgtagggctcgtctccgtcggaacgatgctgatagccccgacagttttaggacaggaggttagtgctagtagtagtacggagagcagtaccactacagctaatactggtaccggtacggcaagtgggatgactgccactactcctagtgctacgacagatactggtgaagcagctgggagcggagctaggagtgaagctaatggtgcatcgtccgtagtatctagcgaagaaagtcagagttcaggcactactccagcctcaccccaagcacagacagctccagcagcaacgtcaacatcatcggtttcttctagtaatgagaaaactcccaagacagcaactacaactacatcatcgactccagtagcaagtaccagtaataatagcaacaaagtaactagtactgaagctgaaacacagacggtggacgtggaacggtatacagttgataaggaaaattcaaagctaaatattaaagacggtaagactccaaaaactaggagtagtgttaataaagacacaaaacttattagaaaccgcgatgacaaacagcgtgatatcgttgatgttactcggacagttgaaactaacgaagatggcctattgatgtttaccggtaacgggttaaaaccgaagcaaattgacgaaggtgccgatgttatggcccttctagatgtctctcaaaagatgacaaaagagaattttgataaggctaaagaacaaataaaaaaaatggttacgactttaacaggcgagccaactgatggtaaggaaaatcataataggcgtaattctgtacgtctaatgactttttaccgtaaggttaatgagcctatcgaattgactgctgaaaatgtagacaaaacattagatgaagtttggaaaaaagctaaagaagattgggactggggcgttgacttacagggagctatccataaagctcgagagatttttaataaggaaaaagaaaaaaaatcgggtaaacgccagcatatcgtcttgttctctcaaggcgaatcaacctttagttatgatattaaaaataaaatgatattacaaaaactacccataactgaaaaagttacgacctctagcccactgtttccttggctccctatctttaatcacactaaccgaaaggcagagattattggtgatttagaaaaagtactagacatggccgaaaaagtgggaataagtttacctagtagtctgaagtcggcagtgaaagtccttggcttaactaatagtgcaataggttctattttagggaaaggtttgacagagtaccttggtttgacagaatatagttcagataacttagatggaggagggtttgattatagtaaacgtgtaggggaaggttactactaccacagtttatcagataggaaatatgaaaatacaatgccccttgaagaagctatcaggacggccttagcatctaattttcccaaactcacagataattggtttttcgatatcttaaatagttttgtcaataaagatacagttgagaaagctaaattagacgtaattatgaaggtacttaatagtattttttacaaaaaagaatatcgctattacaaccataacctgtcagcaatagccgaagctaaaatggctcaacaagagggcattaccttctattccgttgatgttactgatttaaactcagcttctaaaagagtgaggcgacaagcagcagtgcgtaagggcactaaggaagaaaacaaaaaaaatgaagaagagcgtaatacaaagtttgacacctatctgaaaaagatgtctgaaggtaataatttcttaagtaatgttgaagagagagattttttcaaagatactttaacagagctgacaattaaagacgaatttacggataaagttacggtcgagaaggattcctggagtaaatctataactgatggacttaaaaattcaaataacaataatgttaaacatcaacaggcaaacacatcgacatggagtttcttcagttcatccaaagaaagcctcacctggatcatttccaaagaagcactcaaagaaacctttgagaaaaatggttctctcacttttaaatacaaattacgggtcaataaagacaaactattagatttagataagaaagagacaaaacgtgatacatctacggaaaataaaacttctgtaacggcaaatatcatttcaaatactgttaactacaaaattaataatcaagaagttaagggtaacaaacttgatgctgtcaatttgacttatactaaagagaccgttcctgttccagatgtggaaggagaagttgtaccaataccagaaaaaccactggtagaaccaatgacgcctctataccctgcaattcctaattaccctacccctcaacttccaaaagatgaagatctggagattagtggaggtcatggaccgattgtcgatatcgtcgaagatactggtgcaggtgttgagggcggcgctcaaaatggtgtggtttcaactcaggaaaataaagatccaatagttgacatcaccgaagatacccaaccaggtatgtcaggctcaaatgacgcgacagttgtcgaggaagacacagcacctaaacgtccagatgtccttgttggtggtcaaagtgatccaatcgatatcaccgaagatacccaaccaggcatgtcaggttctaatgacgctactgttatcgaggaagacacagcacctaaacgtccagatgtccttgttggtggtcaatgtgatccaatcgatatcaccgaagatacccaaccaggcatgtcaggttctaatgacgctactgttatcgaagatgatacgaaaccaaaacgcttcttccactttgataacgagccacaagcaccagaaaaacctaaagagcaaccatctctcagcttaccacaagctccagtatataaggcagctcatcacttgcctgcatctggagacaaacgtgaagcatcctttacaattgttgctctaacaattattggagctgcaggtttgctcagcaaaaaacgtcgcgacaccgaagaaaactaa

SEQ ID NO: 7 - protein, 1009 aa, Streptococcus pyogenes
STETSTASAGVGTSGTAASETGSGAAVTTATTTTATTNGGPQSTPAVAEATPQPQAQIAPVAAATSTSSASSSSDGKAPQAVTSSTSPSTPAAASSNGSNQEASAETEPQTMEVEKYTVDKENSKLNIKDGKTPKTGSSVNNEKDTKLIRNRDGKLRDIVDVTRTVKTNEDGTIDVTVTVKPKQIDEGADVMALLDVSKKMSEDDFNNAKNKIKKLVKTLTSKSASNSDNDEHKYNSRNSVRLMTFYREISNPIDISGKTEEQLDKLLDDLRKKAKANYDWGVDLQGAIHKAREIFNKEKEKKFGKRRHIVLFSQGESTFSYELQNSVREDKTKLSRLSGAVTSSNPLLPWPPIFNHTHKNIDMLDDVKNLVKLGQTLGIAGLDNLQSTLSLISTGSSLAGAFLGGGSLTEYLTLKEYKSGDLKENQFDYTKRVGEGYHFHSFSERKKTGEIPFKSEIEPKIKELFENNKNNQDKSWTEWIFDKLSLTERIQKAKQETLMKLLEYLFYKREYHYYNHNLSAIAEAKMAQQEGITFYSVDVTDLKTTSKRVKRQVESTEDKKKEKDREDIEKERNEKFDNYLKQMSEGGKDFFEDVDKAEKFKDILTNVTVTETFEDGVNVKDNSWQVSSENNNSLHSNYKSVTHKAASDASWWSLYSNKESLTWTISKEQLKEAFEKNSSLTFKYKLQVNKQKLLDKNKNRTKRDTSTENKTSVTKDIISNTVNYKINNQEVKGNKLDDVKLTYTKETVPVPDVEGEVVPIPEKPLVEPMTPLYPAIPNYPTPDIPTPQLPKDEDLEISGGHGPSVDIVEDTGTGAEGGAQNGVVSTQENRDPIVDITEDTQPGMSGSNDATVVEEDTAPKRPDVLVGGQSDPIDITEDTQPSVSGSNDATVVEEDTVPKRPDSLVGGQSEPIDITEDTQPGMSGSNGATVIEEDTRPKRVFHFDNEPQAPEKPNEQPSLSLPQAPVYKAAHHLPASGDKREASFTIAAPTIIGAAGLLSKKRRDTEGN

SEQ ID NO: 8 - DNA, 3024 bp, Streptococcus pyogenes
gagacgagtactgctagcgctggtgtcggtacgagtgggacggccgccagcgaaactgggagtggagcagccgtaactactgccactactaccaccgctactaccaatggaggaccccagtctactccagcagtagctgaagcgactccacaacctcaagcacagatagctccagtagcagcagcaacgtcgacatcatcggcttcttctagtagtgacgggaaagctcctcaggcagtaacttcatctacatcaccttcaactccagcagcagccagtagtaatggtagcaatcaagaagctagtgctgagactgagccacagacgatggaagtggaaaagtatacagttgataaggaaaattcaaagctaaatattaaagacggtaagactccaaaaactgggagtagtgttaataatgaaaaagacacaaaacttattagaaaccgcgatggcaaacttcgtgatattgttgatgttactcggacagttaaaactaacgaagatggcactattgatgttaccgtaacggttaaaccgaagcaaattgacgaaggtgccgatgttatggcccttttagatgtctctaaaaagatgtcagaagatgattttaacaacgctaagaataagatcaagaaattagtcaaaaccttaacgagtaaatcagcgagtaactcagataatgatgagcataaatataattctcgaaattcggttcgtctgatgaccttttaccgtgagattagcaacccaattgatatatcaggaaaaaccgaggaacaacttgataaattattagacgatcttcgcaaaaaagctaaagctaattatgactggggggttgatttacagggagctatccacaaggctcgagagatttttaataaggaaaaagaaaaaaaatttggtaaacgccggcatatcgtcctattctctcaaggcgagtcaacctttagttatgaacttcaaaatagtgttagagaagataaaactaagttatcccgattaagtggagcagttacttcgtccaaccctctgctaccctggccacctatttttaatcatacgcataaaaatatagacatgcttgacgatgtaaagaatttggtaaaactaggtcaaactttaggaattgcagggctagataatttacagagtacattgagcttaatatcgacaggaagttctctggcaggagcgtttttagggggggggagtctgacagaatacctcactctaaaggagtataaatcaggagacttaaaagaaaatcagtttgattataccaaacgtgttggtgaaggatatcatttccatagtttttctgagagaaaaaaaactggcgaaataccgtttaagagtgaaatagaaccaaaaataaaagaattatttgaaaataacaagaataatcaagataaatcatggactgagtggatatttgataaattatcactgacagagagaattcaaaaagctaagcaggaaacacttatgaagctgcttgaatacctcttttacaaacgtgaataccactactataatcacaacctctcagcgatagctgaagctaaaatggctcaacaagaaggtatcaccttctattccgttgatgttactgatttaaaaacaacttctaaaagagtgaagcgacaagtagaaagtacagaggataagaaaaaagaaaaagatagggaagacattgaaaaagaacgtaacgaaaagtttgataattacttaaaacaaatgtctgaaggcggtaaggatttttttgaagatgttgataaggcagaaaaatttaaagatatcttaactaatgtaacggtgaccgagacttttgaagatggggttaacgttaaggataattcatggcaagtttcatcagagaataataatagcttacatagtaattataagagtgttacacataaagcagcatctgatgcaagttggtggtctttgtatagtaacaaagaaagtcttacttggaccatttctaaagagcagctcaaagaagcctttgagaaaaatagttctctcactttcaagtacaagttacaggtaaataaacaaaaactattagataaaaacaagaatagaacaaaacgtgatacatctacggaaaataagacttctgtaacgaaagacattatttcaaatactgttaactacaaaattaataatcaagaagttaagggtaacaaacttgatgatgtcaagttgacttatactaaagagaccgttcctgttccagatgtggaaggagaagttgtaccaataccagaaaaaccactggtagagccaatgacgcctctatatcctgcaattcctaattacccaacaccagatatccctacccctcaacttccaaaagatgaagatctggagattagtggaggtcatggaccgagtgtcgatatcgtcgaagatactggtacaggtgctgagggcggcgctcaaaacggcgtggtttcaactcaggagaatagagatccaatcgttgacatcaccgaagatacccaaccaggtatgtcaggctcaaatgacgcgacagttgtcgaggaagacacagcacctaaacgtccagatgtccttgttggtggtcaaagtgatccaatcgatatcaccgaagatacccaaccaagtgtgtcaggctcaaatgacgcgacagttgtcgaggaagacacagtacctaaacgtccagatagccttgttggcggtcaaagtgatccaatcgacatcaccgaagatacccaaccaggcatgtcaggctcaaatggcgctactgttatcgaagaagatacgagaccaaaacgcgtcttccactttgataacgagccacaagcaccagaaaaacctaacgagcaaccatctctcagcttaccacaagcgccagtctataaggcagctcatcacttgcctgcatctggagacaaacgtgaagcatcctttacaattgctgctccaacaattattggagctgcaggtttgctcagcaaaaaacgtcgcgacaccgaaggaaactaa

SEQ ID NO: 9 - protein, 806 aa, Streptococcus pyogenes
STETSTASAGVGTSGTAASETGSGAAVTTATTTTATTNGGPQSTPAVAEATPQPQAQIAPVAAATSTSSASSSSDGKAPQAVTSSTSPSTPAAASSNGSNQEASAETEPQTMEVEKYTVDKENSKLNIKDGKTPKTGSSVNNEKDTKLIRNRDGKLRDIVDVTRTVKTNEDGTIDVTVTVKPKQIDEGADVMALLDVSKKMSEDDFNNAKNKIKKLVKTLTSKSASNSDNDEHKYNSRNSVRLMTFYREISNPIDISGKTEEQLDKLLDDLRKKAKANYDWGVDLQGAIHKAREIFNKEKEKKFGKRRHIVLFSQGESTFSYELQNSVREDKTKLSRLSGAVTSSNPLLPWPPIFNHTHKNIDMLDDVKNLVKLGQTLGIAGLDNLQSTLSLISTGSSLAGAFLGGGSLTEYLTLKEYKSGDLKENQFDYTKRVGEGYHFHSFSERKKTGEIPFKSEIEPKIKELFENNKNNQDKSWTEWIFDKLSLTERIQKAKQETLMKLLEYLFYKREYHYYNHNLSAIAEAKMAQQEGITFYSVDVTDLKTTSKRVKRQVESTEDKKKEKDREDIEKERNEKFDNYLKQMSEGGKDFFEDVDKAEKFKDILTNVTVTETFEDGVNVKDNSWQVSSENNNSLHSNYKSVTHKAASDASWWSLYSNKESLTWTISKEQLKEAFEKNSSLTFKYKLQVNKQKLLDKNKNRTKRDTSTENKTSVTKDIISNTVNYKINNQEVKGNKLDDVKLTYTKETVPVPDVEGEVVPIPEKPLVEPMTPLYPAIPNYPTPDIPTPQLPKDEDLEISGGHGPSV

SEQ ID NO: 10 - DNA, 2418 bp, Streptococcus pyogenes
agtactgagacgagtactgctagcgctggtgtcggtacgagtgggacggccgccagcgaaactgggagtggagcagccgtaactactgccactactaccaccgctactaccaatggaggaccccagtctactccagcagtagctgaagcgactccacaacctcaagcacagatagctccagtagcagcagcaacgtcgacatcatcggcttcttctagtagtgacgggaaagctcctcaggcagtaacttcatctacatcaccttcaactccagcagcagccagtagtaatggtagcaatcaagaagctagtgctgagactgagccacagacgatggaagtggaaaagtatacagttgataaggaaaattcaaagctaaatattaaagacggtaagactccaaaaactgggagtagtgttaataatgaaaaagacacaaaacttattagaaaccgcgatggcaaacttcgtgatattgttgatgttactcggacagttaaaactaacgaagatggcactattgatgttaccgtaacggttaaaccgaagcaaattgacgaaggtgccgatgttatggcccttttagatgtctctaaaaagatgtcagaagatgattttaacaacgctaagaataagatcaagaaattagtcaaaaccttaacgagtaaatcagcgagtaactcagataatgatgagcataaatataattctcgaaattcggttcgtctgatgaccttttaccgtgagattagcaacccaattgatatatcaggaaaaaccgaggaacaacttgataaattattagacgatcttcgcaaaaaagctaaagctaattatgactggggggttgatttacagggagctatccacaaggctcgagagatttttaataaggaaaaagaaaaaaaatttggtaaacgccggcatatcgtcctattctctcaaggcgagtcaacctttagttatgaacttcaaaatagtgttagagaagataaaactaagttatcccgattaagtggagcagttacttcgtccaaccctctgctaccctggccacctatttttaatcatacgcataaaaatatagacatgcttgacgatgtaaagaatttggtaaaactaggtcaaactttaggaattgcagggctagataatttacagagtacattgagcttaatatcgacaggaagttctctggcaggagcgtttttagggggggggagtctgacagaatacctcactctaaaggagtataaatcaggagacttaaaagaaaatcagtttgattataccaaacgtgttggtgaaggatatcatttccatagtttttctgagagaaaaaaaactggcgaaataccgtttaagagtgaaatagaaccaaaaataaaagaattatttgaaaataacaagaataatcaagataaatcatggactgagtggatatttgataaattatcactgacagagagaattcaaaaagctaagcaggaaacacttatgaagctgcttgaatacctcttttacaaacgtgaataccactactataatcacaacctctcagcgatagctgaagctaaaatggctcaacaagaaggtatcaccttctattccgttgatgttactgatttaaaaacaacttctaaaagagtgaagcgacaagtagaaagtacagaggataagaaaaaagaaaaagatagggaagacattgaaaaagaacgtaacgaaaagtttgataattacttaaaacaaatgtctgaaggcggtaaggatttttttgaagatgttgataaggcagaaaaatttaaagatatcttaactaatgtaacggtgaccgagacttttgaagatggggttaacgttaaggataattcatggcaagtttcatcagagaataataatagcttacatagtaattataagagtgttacacataaagcagcatctgatgcaagttggtggtctttgtatagtaacaaagaaagtcttacttggaccatttctaaagagcagctcaaagaagcctttgagaaaaatagttctctcactttcaagtacaagttacaggtaaataaacaaaaactattagataaaaacaagaatagaacaaaacgtgatacatctacggaaaataagacttctgtaacgaaagacattatttcaaatactgttaactacaaaattaataatcaagaagttaagggtaacaaacttgatgatgtcaagttgacttatactaaagagaccgttcctgttccagatgtggaaggagaagttgtaccaataccagaaaaaccactggtagagccaatgacgcctctatatcctgcaattcctaattacccaacaccagatatccctacccctcaacttccaaaagatgaagatctggagattagtggaggtcatggaccgagtgtc

SEQ ID NO: 11 - protein, 553 aa, Streptococcus pyogenes
EIEPKIKELFENNKNNQDKSWTEWIFDKLSLTERIQKAKQETLMKLLEYLFYKREYHYYNHNLSAIAEAKMAQQEGITFYSVDVTDLKTTSKRVKRQVESTEDKKKEKDREDIEKERNEKFDNYLKQMSEGGKDFFEDVDKAEKFKDILTNVTVTETFEDGVNVKDNSWQVSSENNNSLHSNYKSVTHKAASDASWWSLYSNKESLTWTISKEQLKEAFEKNSSLTFKYKLQVNKQKLLDKNKNRTKRDTSTENKTSVTKDIISNTVNYKINNQEVKGNKLDDVKLTYTKETVPVPDVEGEVVPIPEKPLVEPMTPLYPAIPNYPTPDIPTPQLPKDEDLEISGGHGPSVDIVEDTGTGAEGGAQNGVVSTQENRDPIVDITEDTQPGMSGSNDATVVEEDTAPKRPDVLVGGQSDPIDITEDTQPSVSGSNDATVVEEDTVPKRPDSLVGGQSEPIDITEDTQPGMSGSNGATVIEEDTRPKRVFHFDNEPQAPEKPNEQPSLSLPQAPVYKAAHHLPASGDKREASFTIAAPTIIGAAGLLSKKRRDTEGN

SEQ ID NO: 12 - DNA, 1662 bp, Streptococcus pyogenes
gaaatagaaccaaaaataaaagaattatttgaaaataacaagaataatcaagataaatcatggactgagtggatatttgataaattatcactgacagagagaattcaaaaagctaagcaggaaacacttatgaagctgcttgaatacctcttttacaaacgtgaataccactactataatcacaacctctcagcgatagctgaagctaaaatggctcaacaagaaggtatcaccttctattccgttgatgttactgatttaaaaacaacttctaaaagagtgaagcgacaagtagaaagtacagaggataagaaaaaagaaaaagatagggaagacattgaaaaagaacgtaacgaaaagtttgataattacttaaaacaaatgtctgaaggcggtaaggatttttttgaagatgttgataaggcagaaaaatttaaagatatcttaactaatgtaacggtgaccgagacttttgaagatggggttaacgttaaggataattcatggcaagtttcatcagagaataataatagcttacatagtaattataagagtgttacacataaagcagcatctgatgcaagttggtggtctttgtatagtaacaaagaaagtcttacttggaccatttctaaagagcagctcaaagaagcctttgagaaaaatagttctctcactttcaagtacaagttacaggtaaataaacaaaaactattagataaaaacaagaatagaacaaaacgtgatacatctacggaaaataagacttctgtaacgaaagacattatttcaaatactgttaactacaaaattaataatcaagaagttaagggtaacaaacttgatgatgtcaagttgacttatactaaagagaccgttcctgttccagatgtggaaggagaagttgtaccaataccagaaaaaccactggtagagccaatgacgcctctatatcctgcaattcctaattacccaacaccagatatccctacccctcaacttccaaaagatgaagatctggagattagtggaggtcatggaccgagtgtcgatatcgtcgaagatactggtacaggtgctgagggcggcgctcaaaacggcgtggtttcaactcaggagaatagagatccaatcgttgacatcaccgaagatacccaaccaggtatgtcaggctcaaatgacgcgacagttgtcgaggaagacacagcacctaaacgtccagatgtccttgttggtggtcaaagtgatccaatcgatatcaccgaagatacccaaccaagtgtgtcaggctcaaatgacgcgacagttgtcgaggaagacacagtacctaaacgtccagatagccttgttggcggtcaaagtgatccaatcgacatcaccgaagatacccaaccaggcatgtcaggctcaaatggcgctactgttatcgaagaagatacgagaccaaaacgcgtcttccactttgataacgagccacaagcaccagaaaaacctaacgagcaaccatctctcagcttaccacaagcgccagtctataaggcagctcatcacttgcctgcatctggagacaaacgtgaagcatcctttacaattgctgctccaacaattattggagctgcaggtttgctcagcaaaaaacgtcgcgacaccgaaggaaactaa

SEQ ID NO: 13 - protein, 456 aa, Streptococcus pyogenes
STETSTASAGVGTSGTAASETGSGAAVTTATTTTATTNGGPQSTPAVAEATPQPQAQIAPVAAATSTSSASSSSDGKAPQAVTSSTSPSTPAAASSNGSNQEASAETEPQTMEVEKYTVDKENSKLNIKDGKTPKTGSSVNNEKDTKLIRNRDGKLRDIVDVTRTVKTNEDGTIDVTVTVKPKQIDEGADVMALLDVSKKMSEDDFNNAKNKIKKLVKTLTSKSASNSDNDEHKYNSRNSVRLMTFYREISNPIDISGKTEEQLDKLLDDLRKKAKANYDWGVDLQGAIHKAREIFNKEKEKKFGKRRHIVLFSQGESTFSYELQNSVREDKTKLSRLSGAVTSSNPLLPWPPIFNHTHKNIDMLDDVKNLVKLGQTLGIAGLDNLQSTLSLISTGSSLAGAFLGGGSLTEYLTLKEYKSGDLKENQFDYTKRVGEGYHFHSFSERKKTGEIPFKS

SEQ ID NO: 14 - DNA, 1365 bp, Streptococcus pyogenes
actgagacgagtactgctagcgctggtgtcggtacgagtgggacggccgccagcgaaactgggagtggagcagccgtaactactgccactactaccaccgctactaccaatggaggaccccagtctactccagcagtagctgaagcgactccacaacctcaagcacagatagctccagtagcagcagcaacgtcgacatcatcggcttcttctagtagtgacgggaaagctcctcaggcagtaacttcatctacatcaccttcaactccagcagcagccagtagtaatggtagcaatcaagaagctagtgctgagactgagccacagacgatggaagtggaaaagtatacagttgataaggaaaattcaaagctaaatattaaagacggtaagactccaaaaactgggagtagtgttaataatgaaaaagacacaaaacttattagaaaccgcgatggcaaacttcgtgatattgttgatgttactcggacagttaaaactaacgaagatggcactattgatgttaccgtaacggttaaaccgaagcaaattgacgaaggtgccgatgttatggcccttttagatgtctctaaaaagatgtcagaagatgattttaacaacgctaagaataagatcaagaaattagtcaaaaccttaacgagtaaatcagcgagtaactcagataatgatgagcataaatataattctcgaaattcggttcgtctgatgaccttttaccgtgagattagcaacccaattgatatatcaggaaaaaccgaggaacaacttgataaattattagacgatcttcgcaaaaaagctaaagctaattatgactggggggttgatttacagggagctatccacaaggctcgagagatttttaataaggaaaaagaaaaaaaatttggtaaacgccggcatatcgtcctattctctcaaggcgagtcaacctttagttatgaacttcaaaatagtgttagagaagataaaactaagttatcccgattaagtggagcagttacttcgtccaaccctctgctaccctggccacctatttttaatcatacgcataaaaatatagacatgcttgacgatgtaaagaatttggtaaaactaggtcaaactttaggaattgcagggctagataatttacagagtacattgagcttaatatcgacaggaagttctctggcaggagcgtttttagggggggggagtctgacagaatacctcactctaaaggagtataaatcaggagacttaaaagaaaatcagtttgattataccaaacgtgttggtgaaggatatcatttccatagtttttctgagagaaaaaaaactggcgaaataccgtttaagagt

SEQ ID NO: 15 - protein, 19 aa, Streptococcus pyogenes
ETEPQTMDVEQYTVDKENS

SEQ ID NO: 16 - protein, 21 aa, Streptococcus pyogenes
DIFDVKREVKTNGDGTLDVLT

SEQ ID NO: 17 - protein, 20 aa, Streptococcus pyogenes
PKQIDEGADVMALLDVSQKM

SEQ ID NO: 18 - protein, 16 aa, Streptococcus pyogenes
FDKAKEQIKKLVTTLT

SEQ ID NO: 19 - protein, 14 aa, Streptococcus pyogenes
YNRRNSVRLMTFYR

SEQ ID NO: 20 - protein, 20 aa, Streptococcus pyogenes
WGDVLQGAIHKAREIFNKEK

SEQ ID NO: 21 - protein, 19 aa, Streptococcus pyogenes
RQHIVLFSQGESTFSYDIK

SEQ ID NO: 22 - protein, 16 aa, Streptococcus pyogenes
TTSNPLFPWLPIFNHT

SEQ ID NO: 23 - protein, 19 aa, Streptococcus pyogenes
FDYSKRVGEGYYYHSFSDR

SEQ ID NO: 24 - protein, 18 aa, Streptococcus pyogenes
ERNEKFDNYLKEMSEGGK

SEQ ID NO: 25 - protein, 15 aa, Streptococcus pyogenes
DVDKADKFKDTLTEL

SEQ ID NO: 26 - protein, 12 aa, Streptococcus pyogenes
TKESLTWTISKD

SEQ ID NO: 27 - protein, 15 aa, Streptococcus pyogenes
SLTLKYKLKVNKDKL

SEQ ID NO: 28 - protein, 102 aa, Streptococcus pyogenes
DITEDTQPGMSGSNDATVVEEDTAPQRPDVLVGGQSDPIDITEDTQPGMSGSNDATVVEEDTVPKRPDILVGGQSDPIDITEDTQPGMSGSNDATVIEEDTK

SEQ ID NO: 29 - protein, 34 aa, Streptococcus pyogenes
GASSVASSASSSSNGSVASSSEPQMPQAQTAPQM

SEQ ID NO: 30 - protein, 246 aa, Streptococcus pyogenes
IAPTVLGQEVSTTGSTETSAASTTTNTASTVETSTTTGTSVTAASEASSESSDASVVSSGGRQTSESAQASKQPQAQTAVASSSSSSKANESSSSASDVKAPKAVSTTSSSATVASPSNGSNKEANAETEPQQMMEVEKYTVDKENSELKVKDGTQPKKGSTVNENTKLIRNRDGKQRDIVDVTRTVKTNEDGTIDVTVTVKPKQIDEGADVMALLDVSKKMTQENFDKAKEQIKKMVTTLTSKTD

SEQ ID NO: 31 - protein, 923 aa, Streptococcus pyogenes
IAPTVLGQEVSANTETSTTPATTTPSAGTGTATTSGTATTTPSATTDAGGAAGSGTNGASSVTSSGGSQSSESAQASPQAQAAPAAAETTPKAQAQTATVASASTTASSSSSDGKAPQAASTTSSSTPAVASNNSNQEAGTEAETPMMEVEQYTVDNKATELNIKDGKNLKNGSRVVDKNTKLIRNRDGEQRDIVDIKREVKTNADGTIDVTVTVTPKEIDEGADVMALLDVSKKMTDADFKNAKDKIKKLVTTLTSNSDNAEHKHNSRNSVRLMTFYREISDPIDISGKTDAELDKILNDLREKAKANYDWGVDLQGAIHKAREIFKKDQEKKSGKRQHIVLFSQGESTFSYDINDKTKLKTITEDKITTSNPLFPWLPIFNHTNRKADMLDDIAKVIKKVKGLGVESVGTAESVLSALTALNKLGSLLTGSMTEYITLKEYDSDKLGAERFDYTKRVGEGYYYHSFSDRKSEDTMFFSDRKSEDTMPFESEIMAGLKSHLPKFKEGDWFTNVLQYFGLKEKAEQAKLDVIMKVIKSVFYKRQYHYYNHNLSAIAEAKMAQEEGITFYSVDVTDLKTTSTRVKRQTAVYKDDKKKEIEERNNKFDKYLKEMSEGKTFLEDKDVTNKDKFKDTLTELTIKDEFSDKVKVEENSWNKPVADELKNSNKNSITHQKASSWFLRSTKESLTWTISKDQLKKAFEDGKPLTLTYKLKVDNNKFKTALEEEKKKRAKRSTPTENENSVTEKIISNTITYQINQKKGTDKSLGDVKLTYSKLKVPVPQIDGHVIEPQAPTLPKLPPVIEHGPNFEYEEETGYQLPLKHGSNAPDTQVTIEEDTVPQRPDILVGGQSGPVDITEDTQPGMSGSNDATVVEEDTAPKRPDVLVGGQSDPIDITEDTQPSVSGSNDATVVEEDTVPKRPDIL

SEQ ID NO: 32 - protein, 454 aa, Streptococcus pyogenes
IAPTVLGQEVSTTGSTETSAASTASVDATTSGTTASGASGESSDASVASSEGSQGSESAPASPQPQPQAQTAPAATSASSKAKTEEQTPKAATSSTPSTPAASSSSNSNQEASAETEPQMMDVEKYTVDKESSELKVKDGKKPKNENKVDKDTKLIRNRDGEQRDIFDIKREVKTNADGTIDVTVTVTPKEIDEGADVMALLDVSQKMTKENFDKAKEQIKKMVTTLTGEPTDGKENRNRRNSVRLMTFYRKISEPIDLSGKTSEEVEKELDNIWDKVKKEDWDWGVDLQGAIHKARDIFKKEKESKKRQHIVLFSQGESTFSYDINDKDKNNTVRKNRITGKVTTSNPLFPWLPIFNHTNQKAEVIDDVDKLLDFAEKMGISLPKGLRAGVQAIGLSNSFLSTFTGSGLTEYLTLNEYGSDILKEKQFDYTKRVGEGYYYHSYSKRTHGDKMP

SEQ ID NO: 33 - protein, 409 aa, Streptococcus pyogenes
ETSTTTSTSGTAASGAGSESSDASVVPSEGSQSSGTTTPASKQPQAQTAPAATSASSTSSSSSDGKAPQAATISTSSTPAAGTSSNSNQVTGTEAEPQTMDVERYTVDKENSKLNIKDGDKPKNRSSVDKDTKLIRNRDGKQRDIVDVTRTVKTNEDGTIDVTVTVKPKQIDEGADVMALLDVSKKMSEDDFNNAKDKIKKLVTTLTSKSANGQQNLNNRNTVRLMTFYRKISDPIDLSGKTSEEVEEELNKIWDKVKTKDWDWGVDLQGAIHKARDIFKKEKESKKRQHIVLFSQGESTFSYELHNSVKEDKYKLSRLTETVTSSNPLLPWPPIFNHTHKNIDMLDDVKHLIKLGQALGIKELDSLQSTLKLVSAGSNAAGLLLGGGSLTEYLTLKEYKSGNLTENQF

SEQ ID NO: 34 - protein, 232 aa, Streptococcus pyogenes
IAPTVLGQEVSTSASSTETSANTNTNTSTASAGTGTSGTASTTPSVGTSTGGAAGGEAAVASSGGSQSSDTTPASPQAQTSEQPAATSTSSNSSSDGQTPKTATTSPSTPVVANSNGNQVTGTEASPQMMDVEKYTVDKESSELNIKDGKTPKNGISVTKDTKLIRNRDGKQRDIVDVTRTVKANEDGTIDVTVTVKPKQIDEGADVMALLDVSKKMTQENFDKAKEQIKKL

SEQ ID NO: 35 - protein, 250 aa, Streptococcus pyogenes
IAPTVLGQEVSTTGSTETSAASTASPGTGTATTSVTAASGAGSEATEATATTTNGGPQSATVTSEATPKAQAQTSEQPAATSASSTSSSKAKTEEQTPKAATSSTPSTPAASSSSNSNQGASTETEPQMMEVEQYKVDKEETELKVKDGNQPKNERSVSQNTKLIRNRDGEQRDIVDIKREVKDNGDGTLDVTLKVTPKEIDKGADVMALLDVSQKMTDADFDNAKEKIKKLVTTLTSKSNSDEHKHNSR

SEQ ID NO: 36 - protein, 197 aa, Streptococcus pyogenes
IAPTVLGQEVSTETSTTSTASTTTGTSGTATTTPSAITGTDGAAGSGTSDVSVVSSEGSQSSESAQASPQAQTATVASASTTASPSSSSASDGKAPQAASTTSSSATVANPSNGSNQVTGTEVEPQMMDVEQYKVNKEKTELTVKDDKQQLKIRKDVDELKNKDLFDVKREVKDNGDGTLDVTLKVMPKQIDEGADV

SEQ ID NO: 37 - protein, 873 aa, Streptococcus pyogenes
IAPTVLGQEVSANTETSTTPATTTPSAGTGTATTSGTATTTPSATTDAGGAAGSGTNGASSVTSSGGSQSSESAQASPQAQAAPAAAETTPKAQAQTATVASASTTASSSSSDGKAPQAASTTSSSTPAVASNNSNQEAGTEAETPMMEVEQYTVDNKATELNIKDGKNLKNGSRVVDKNTKLIRNRDGEQRDIVDIKREVKTNADGTIDVTVTVTPKEIDEGADVMALLDVSKKMTDADFKNAKDKIKKLVTTLTSNSDNAEHKHNSRNSVRLMTFYREISDPIDISGKTDAELDKILNDLREKAKANYDWGVDLQGAIHKAREIFKKDQEKKSGKRQHIVLFSQGESTFSYDIKNKNDSKLKKARLTTVTTSNPLFSWFPIFDRTNRKADMLDGFDKLLSIAQKFGVEIPNGLKTGLKAAATTNSLLSSFTGGDGLTDYLTLREYMADKLQETDFNYSNRVGEGYHHHSFSERNTHDMPMKETLEKLLDSQIPRLDKESWFGWALDKLSLTETYQNGQKVALMKILDYLFYKREYVYYNHNLSAIAEAKMAQQEGITFYSVDVTDFETTSKRVKRQVGVLQETAKKEPEKERNDKFDKYLEDMSEGKKFLKDIDNQDKFKDILTDVTVTETFEGQVAAGSDSWSNSHGVVKYQKNENGGWFTTSKKESLTWTISKEQLKKAFEDGKPLTFTYKLKVEKDKFKTALEENKKQRTKRSAPTENENSVTKKIISNTVTYKINNQEVKDNNLDEVNLTYSKLKVPVPQIDGQVIEPQAPKLPELPPVTERGPVLDYTEESIYRLPLEHGSNAPDTQVTIEEDTVPQRPDILVGGQSGPVDITEDTQPGMSGSNDATVVEEDTTPKRPDVLVGGQS

SEQ ID NO: 38 - protein, 245 aa, Streptococcus pyogenes
IAPTVLGQEVSASTTSSTETSATTSTSTGTSETAASEAGSGASDVSIASSGGSQSSGTTPSATTGTGEAAGSGTTVATATTTNGGTQSTPAAASASSTSSTSSTSSSEDKAPKAASTTLSSATVASPSNGSNQEASAETAPQMMDVERYEVDNKETELKVKDGKETNGSGVSKKLIRNRDDEQRGIVDVKREVKTNSDGTIDVTVTVKPKQIDEGADVMALLDVSKKMTEEDFKNAKDKIKKLVK

SEQ ID NO: 39 - protein, 1029 aa, Streptococcus pyogenes
MTNCKYKLRKLSVGLVSVGTMLIAPTVLGQEVSTGVSNTEASASSTNTNTASADATASGTAATTPSAGTSTSTGEAAGSGLSSEANWSDAAVASSGGSQSSGTTPASPQAQTAPAATTTTSSASSSNEKPLKTATTTTSSTPAASSSSNGNQVTGTEVEPQMMDVEQYKVDKENSELTVKVDRRQLKIRKDVDNPKDKDLFDVKREVKDNGGGTLDVTLKVMPKQIDGGADVMALLDVSKKMTQENFDKAKGQIKKVVTTLTGESTDGKGNYNRRNSVRLMTFYRKVSDPIELTTKTIGAKLEEVWEQAKKDWDWGVDLQGAIHRARDIFRGEKGSKGRQHIVLFSQGESTFSYDISDKDNGASVRVPSITGNVTASNPLFPWLPIFPPTPHPAEVIDDVDKLLGFAENLGISLPKGLREGVTAIGLRRGLLSSFTGSGLTEYLTLSEYGSAILYYAQFDYTTRVGEGYYYHSYSVRTHGDMLPFESEIRKALEQVLPKIEDREWAPMFIDIFGLPIQKVNQSGIDVIMKVINSIFYSRQYFYYNRNLSAIAEAKMAQEEGITFYSVDVTDLSSASKRAKRQTAVPQKTTKKESEEDRNNKFDGYLKKMSEGGKEFFTGVDKADKFKDTLTELTIKDEFEDKVTVETNSEGKKNYKTNLKGNTLKVNHTPSKAGSLSWFSSATKESLTWTISKDLGRKKAFEDGKPLTLTYKLKVDNGKFKKSLEENNKKRTKRSAPTENENSIKEKIISNTITYKINNQKGQTGKKLDDVSLTYSKLKVPVPQIDEKVIEQQEPTLPKLPPVIEHGPNFEYEEETGYQLPLKHGRNAPDTQVTIEEDTVPQRPDILVGGQSDPIDITEDTQPGMSGSNDATVVEEDTAPKRPDVLVGGQSEPIDITEDTQPSVSGSNDATVVEEDTVPKRPDILVGGQSDQIDITEDTQPGMSGSNDATVIEEDTKPKRFFHFDNEPQAPLKPYEQPSLSLPQAPVYKAAHHLPASGDKREATITIVALTLIGAAGLLSKKRRDTEEN

SEQ ID NO: 40 - protein, 258 aa, Streptococcus pyogenes
IAPTVLGQEVSTGASSSTETSASSNNTNTASTVETSTTTSTSGTAASGTGSEAAVASSGGSQSSGTTPASPQAQTSEQPAVTSASSTSSSSEEKTPKAANTASSSATVASPSNGSNQEASAETEPQTMEVEKYTVDRENSELKVKDGTQPKKGRSVSQDTKLIKNRDGKQRDIVDVTRTVKTNEDGTIDVTVTVKPKQIDEGADVMALLDVSKKMTEDDFKNAKEKIKKLVTTLTSKSPDGQPNHNARNSVRLMTFYH

SEQ ID NO: 41 - protein, 216 aa, Streptococcus pyogenes
IAPTVLGQEVSTTSTASTETSASSTASASTDTATTSVTAATTPSTTTGTSVTAANGTSSGTTVATATTTNGGTQSTPAAAETTPKPQAQTAVATSSSSSNANPLSETQASKAASTTSSSATVASSSNGSNQGAGTEAEPQMMDVEKYTVDKENSELKVKDGKETNGSGVNKKLIRNRDGEQRDIFDIKREVKTNSDGTIDVTVTVTPKEIDEGADV

SEQ ID NO: 42 - protein, 235 aa, Streptococcus pyogenes
IAPTVLGQEVSASTTSTETSATSTNTSTASAGTSTSGTATTTSSATTDAGRAAGSGTASGTNGVSSVASSEGSQGSEPGQASTQPQAQTLEQSAATSTSSASSSNEEKSIKSATSSTPSTAAASSSSNGNQEASAGTAPQMMEVERYTVDKENSELKVKDGDKLKNGGSATKETKLIRNRDGKQRDIVDVTRTVKTNEDGTIDVTVTVKPKQIDEGADVMALLDVSQKMTKENFD

SEQ ID NO: 43 - protein, 865 aa, Streptococcus pyogenes
IAPTVLGQEVNASTTSSTETSASSAASVSAGTGTSGTAAREAGSGASDESSDASVASSEGSQGSKFAPASPQPQAQTATVASASTTASPSSSSASDGKAPQAASTKSSSATVASSSNGSNQGAGAEDAPQMMDVEQYTVDKESSELKVKDGKNPKNGSRADKNTKLIRNRDDEQRDIFDIKREVKDNGDGTLDVTLKVTPKEIDEGADVMALLDVSQKMTDADFKNAKDKIKKLVTTLTSKSNSDEHKHNSRNSVRLMTFYREISDPIDISGKTEAELDQLLNELREKAKANYDWGVDLQGAIHKTREIFNKEQKSKKRQHIVLFSQGESTFSYDIQKSEKEKDSNLSRINEKITSSNPLLPWPPIFDHTHQNADMLKDVEFLISLAQKLGVTGLSSIKTILQGVGLANQFGGLLLGGGSLTEYLTLQEYKTNTFTKEQFDYTKRVGEGYHYHSFSTRKSEDKIPFEKDIEAALKTALPESKSENWFTKVLTYFGLKNKAEQAKLDVIMKVIKSVFYKRQYHYYNHNLSAIAEAKMAQKDGITFYSVDVTDSDNASKRVKRQVGKEQSKKKKEDAGKDRSKKFDDYLKKMSEGDNFLSNVEERDKFKDTLTELTVKDEFSDKVTVQNNSEGKKYQVTGLINDIKVSYTAANNTGWFTRTKESLTWTISKEQLKKAFEDGKPLTLTYKLKVDNDKLKKALDDKRKDRKKRDTSTKNENSVTERIISNITTYKINGQEVKDNNLSDVSLTYSKLKVPVPQIDGHVIEPQAPTLPKLPPVTERGPVLDYTEESIYRLPLEHGSNAPDTQVTIEEDTVPQRPDILVGGQSGPVDITEDTQPGMSGSNDATVVEEDTAPQRPDVLVGGQS

SEQ ID NO: 44 - protein, 214 aa, Streptococcus pyogenes
IAPTVLGQEVSAGASSSTETSAASASAGTSTSETAASGTGSEAAVVSSEGSQSSESAQASPQPQPQAQTVTATTSTSSTSSSSDGKSTKSATSSTSSAFSTSSSEDKAPKAASTKSSSTTVASPSNGSNQGASTETEPQMMEVEQYKVDKEETELKVKDGSKLNSSSDKKLIRNRDGEQRDIFDIKREVKTNSDGTIDVTVTVTPKEIDEGADV

SEQ ID NO: 45 - protein, 1013 aa, Streptococcus pyogenes
MTNCKYKLRKLSVGLVSVGTMLIAPTVLGQEVSTTGSTETSAASTTTSTASTVETSTTTGTSVTAASEASSESSDVSVVSSEGSQSSASAPASPQPQAQTPPAATSTSSASSSSSEDKASKAATSSTSSSTPAVASSSSNSNQATGTEVEPQMMEVEQYTVNKESSELKVKDGKEMNGSGVSKKLIRNRDGEQRDIFDIKREVKTNADGTIDVTVTVTPKEIDKGADVMALLDVSKKMSKEDFNNAKTKIKQLVKTLTEKNGENHNSRNSVRLMTFYREISDPIDISGKTEEQLDKILNDLRKKAKANYDWGVDLQGAIHKAREIFKRDQEKKSGKRQHIVLFSQGESTFSYDIKNKNDSTVTKTRITEKVTTSNPLLPWPPIFDHTHQNADMLEDSAKLIKKLKSLGLESLQTADNILQALQAANRIGSLFGKSPTEYLTLNEYDSNKLGEESFDYSKHVGEGYYYHSFSDRKSENTMPLESAIKTALTSNFPKIPDSWFFGILKSSDIKAKVEKAKLDVIMQVLKSIFYKREYRYYNHNLSAIAEAKMAQKDGITFYSVDVTSPNQPATTKRSRRSTEKKEAEERNEKFDKYLKEMSEGGKKFFNDVDKTDKFKDTLTELKIKDEFTDKVTVEENSWNTLSTAGLKNSNKNKDVQHQKASQPSVWSFTSPSKESLTWTISKEQLKEAFEKNGSLTFKYKLRVNKDKLLDKNKNITKRDTSTEDKTSVTANIISNTITYKINDQEVKGNNLDDVNLTYSKFKVPVPQIDGHVIEPQAPTLPKLPPVIEHGPNFEYEEETGYQLPLKHGSNAPDTQVTIEEDTVPQRPDILVGGQSGPVDITEDTQPGMSGSNDATVVEEDTAPQRPDVHVGGQSDPIDITEDTQPGMSGSNDATVVEEDTVPKRPDVHVGGQSDPIDITEDTQPGMSGSNDATVIEEDTKPKRFFHFENEPQAPEKPKEQPSLSLPQAPVYKAAHHLPASGDKREASFTIVALTIIGAAGLLSKKRRDTEEN

SEQ ID NO: 46 - protein, 202 aa, Streptococcus pyogenes
IAPTVLGQEVSTGASSTETSASSNNTNTNTASTVETSTTTSTSGTAASGTGSEAAVASSGGSQSSGTTPASPQAQTSEQPAVTSASSTSSSSEEKTPQAANTASSSATVASPSNGSNQEASAETEPQMMEVEQYKVDKEETELKVKDGNKLNNSSDKKLIRNRDGEQRDIFDIKREVKTNSDGTIDVTVTVTPKEIDEGADV

SEQ ID NO: 47 - protein, 256 aa, Streptococcus pyogenes
IAPTVLGQEVNASTETSTTSTSTASVDATTSGTAATTPSAGTSTSTGEAAGSGASSEANGASSVVSSEESQSSGTTPASPQAQTAPAATSTSSASSSNEKTPKTATTTTSTSSTPVASTSNNSNKVTSTEAETPMMDVEQYTVDKKDSSVTQTDDKKLLKIRRDGDEKTRDLYDVKREVKDNGDGTLDVTLKVTPKQIDEGADVMALLDVSKKMTETDFKNAKEKIKKLVTTLTSKSTDNQPNHNARNSVRLMTFY

SEQ ID NO: 48 - protein, 208 aa, Streptococcus pyogenes
IAPTVLGQEVSTGTASTETSASSTNSNNTASADATASGTAASGTASGTNGAFSVTSSEGSQSSESAPASKQPQAVVSTAATSASTASSSSSEEKTPKAATASTTASSTPATSSSNDGNNQGASTEVETPMMEVEQYKVNKEKTELTVKDGTQPKNGKTANQNTKLIRNRDGEQRDIFDIKREVKTNADGTIDVTVTVTPKEIDEGADV

SEQ ID NO: 49 - protein, 221 aa, Streptococcus pyogenes
IAPTVLGQEVNASTETSASSTTSTASTAETSTPTGTSGTAASGASGEATVATANGGPQSAPATSEATPQPQAQAAPAASAPTTVTSSSSSDSDAKTPKAASTTSSSATVASPSNGSNKEANAETAPQMMDVEQYKIKDENSSITVADKAKQLKIRRDDNPKDKDLFDVKREVKDNGDGTLDVTLKVMPKQIDEGADVMALLDVSQKMTKENFDKAKEQIKK

SEQ ID NO: 50 - protein, 197 aa, Streptococcus pyogenes
IAPTALGQEVSTNTNTSTASAGTTANGTADTIPNATTDAGGAAGSGTNGASSVTSSGGSQSSESAQASPQAQTATVASASTTASPSSASASDVKAPRAATSSTPSTPAASTSSNSNQVTGTEAEPQMMDVEQYTVDKKDSSVTQTDNKKLLKIRRDGKEKEDRTLYDIKREVKDNGDGTLDVTLKVTPKQIDEGADV

SEQ ID NO: 51 - protein, 447 aa, Streptococcus pyogenes
IAPTILGQEVSASTETSTTSTSTASVDATTSGTAATTPSASTSTGGTAASGASGEATVATANGGPQSAPATSEATPQPQAQTATVVSASTTASPSSASDVKAPQAASTTSASSTPAAASNNSNQATGTEVETPMMEVEQYKVDKEKTELKVKDGNKLNSSGSDKQLIRNRDGKQRDIVDVTRTVKTNEDGTIDVTVTVKPKQIDEGADVMALLDVSKKMSEDDFKNAKEKIKTLVTTLTGKSSDGKENLNNRNTVRLMTFYRKISEPIDLSGKTSEEVEKELDNIWDKVKKEDWDWGVDLQGAIHRARDIFKKDQEKKSGKRQHIVLFSQGESTFSYDIHEKSKNLSRINEKITSSNPLLHWPPIFNHTHQNADMLNEINSIVKIGEQLGIKGLSNIRDILTAAGVGSGLLGSVVGGGSLTEYLTLKEYKSDKLLEESQFDYTQTCG

SEQ ID NO: 52 - protein, 247 aa, Streptococcus pyogenes
IAPTVLGQEVNANAETSTTPATTTPSTSTITSGTAASVTGNEATVATATTTNGGTQSVTATSEATPQPQAQKAPATTSTSSASSSNEKSTTAATSSTPSTSSSSEANSDAKSNKVAATPPSATVASPSNGSNQGTSAETAPQMMEVEQYKIKDENSSITVADKDKQLKIRRDIDNPKDKDLFDVTREVKDNGDGTLDVTLKVTPKQIDEGADVMALLDVSKKMSEDDFKNAKEKIKKLVTTLTSKSA

SEQ ID NO: 53 - protein, 213 aa, Streptococcus pyogenes
IAPTVLGQEVSATGSTETSAASTASPGTTANGTADTTPSATTGTGEAAGSGTSSGTTVATATTTNGGTQSTPAAAETTPQPQAQTATVASASTTASSSSSDGKAPQAASTTSSSTPAAASNNSNQEASAKAETPMMDVEQYKVDKEETELKVKDGDKSKNGRTVNQNTKLIRNRDGKQRDIFDIKREVKDNGDGTLDVTLKVTPKQIDEGADV

SEQ ID NO: 54 - protein, 430 aa, Streptococcus pyogenes
IAPTVLGQEVGASTTNTETSASTTSTAETSTTTGTSGTAASETGSGTSDVSVVSSEGSQGSESAQASPQAQAAPAAETTPKAQAQAAPVASASTTASSASSNVKTPKTESATISSTPAVASSNGSNQEASAETEPQMMDVEQYKVNKEKTELTVKDGTQPKNGRTVNQNTKLIRNRDGEQRDIFDIKREVKTNADGTIDVTVTVTPKEIDEGADVMALLDVSKKMTEEDFKNAKDKIKKLVTTLTGDKREASFTRNSVRLMTFYREISDPIDISGKTDDELDKLLDKLRQEAKDECDWGVDLQGAIHKAREVFNNENNNSKKKSGKRQHIVLFSQGESTFSYDIKNKKDSKLQKNRLTTVTTSNPLFSWFPIFDHTNRKADMLEDFDKLLSIAQKFGIEIPKEVTAGLRAVTTANSWFGSVIGSDSLTDY

SEQ ID NO: 55 - protein, 456 aa, Streptococcus pyogenes
IAPTVLGQEVSATGSTETSAASTASPGTTANGTADTTPSATTGTGEAAGSGTSSGTTVATATTTNGGTQSTTASETTPQPQAQTAVATSSSSSNANASSSSEEKTPKTATSSTSSTSSTPAAASNNSNQEASAETEPQMMDVEQYKVDKEETELKVKDGDKSKNGRTVDQNTKLIRNRDGKQRDIVDVTRTVKTNEDGTIDVTVTVKPKQIDEGADVMALLDVSKKMTDADFNNAKDKIKKLVTTLTSKSPDGQQNLNNRNRVRLMTFYREISDSIDISGKTDDELDGLLNKLRQEAKDEYDWGVDLQGAIHKAREIFNKEKEKNSGKRQHIVLFSQGESTFSYDIQKSEKENSRNLSRINEKITSSNPLLPWPPIFNQTHQNADMLKDVDFLISLAQKLGMTELSSIKTILQGVGQVSQFGGFLLGGGSLTEYLTLQEYKTDTFTKEQFDYTKTR

SEQ ID NO: 56 - protein, 1091 aa, Streptococcus dysgalactiae
MTNCKYKLRKLSIGLVSVGTMFMAAPVMGEDASQPTASVTTESPAIQTEEDQGSQAEALEEPTPAPQTSPSTVSAVPAEAAAMADEKGIAEAPAHEPAPKASVQAEAASPAGKAEATTNTGQPTNTEQARSRSKRAAEIAPQTIEVEKLEVDKENSSLTVKDGEKDKQLIKHRDGNQRDIFDISRDVKVNQDGTMDVTLTVKPKQIDEGAEVIVLLDTSQKMTETDFNTAKENIKKLVTTLTGTTDKEGKNVSHYNNRNSVRLIDFYRKVGESTDLSGWDAKKIDEKLNEVWKKAKDDYNGWGVDLQGAIHKAREIFNLDKEKRSGKRQHIVLFSQGESTFSYDIKDKSKMDKVAVEEPVTYSNPLFPWPFYFDTTTRTHNVVNDAKKLIDFLNKLGISQFNGAVDNVATVGNTLLGLGSFFGLKNPLDYISLADLETSKLNSEKFDYSRRVGEGYNFRSYFDREVDKVGFKKILVEKIKGNLKKFQPKQTDTWLSSLGLNSIKEKIQDWMIDKALDNLFYRRQYQFYNHNLSAQAEARMAREEGIKFYAVDVTEPERIAKEINSQKYSEAYTNHLKKKAEEARELAKKRNEKFDKYLKEMSESQKFFKDVEDPEKFKDILTELKVTETFEEKVSVNNSEQRKSNKEVEYKKASSNSSFLSFIFSSSTNESITWTLSKDKLQKALQSGETLTLEYKLKIHKDKFKLAPQTRSKRSLDTSENKKSVTEKVITSDVKYKINDKEVKGKELDDVSLTYSKETVRKPQVEPNVPDTPQEKPLTPLAPSEPSQPSIPETPLIPSEPSVPETSTPEGPTEGENNLGGQSEEITITEDSQSGMSGQNPGSGNETVVEDTQTSQEDIVLGGPGQVIDFTEDSQPGMSGNNSHTITEDSKPSQEDEVIIGGQGQVIDFTEDTQSGMSGDNSHTDGTVLEEDSKPSQEDEVIIGGQGQVIDFTEDTQTGMSGAGQVESPTITEETHKPEIIMGGQSDPIDMVEDTLPGMSGSNEATVVEEDTRPKLQFHFDNEEPVPATVPTVSQTPIAQVESKVPHAKAESALPQTGDTNKLETFFTITALTVIGAAGLLGKKRRNNQTD

SEQ ID NO: 57 - DNA, 3276 bp, Streptococcus dysgalactiae
atgactaactgtaagtataaactacggaaattatctattggtcttgtttcggttggaaccatgtttatggcagcacctgttatgggagaggacgcttctcaaccaactgcttctgttactacggaatccccagcgatacaaactgaagaggatcaaggtagccaagctgaggcgctagaagaaccgacaccagctcctcaaactagtccttctacagtaagcgctgtgccagctgaagcagctgccatggctgatgagaaagggattgctgaagccccagcccacgagccagctccaaaagcttctgttcaagcggaagcggctagccccgctggtaaagctgaagctactactaacactggtcaaccgaccaacacagagcaagcacgttcccgcagcaagcgtgccgcagagatagcacctcaaaccatagaagtggaaaaacttgaggttgataaagaaaactccagccttactgttaaagatggtgaaaaagacaaacagcttattaaacacagagatggcaatcagcgggatatttttgatatcagtcgagatgtgaaagtcaatcaagatggaacaatggatgttaccctaacagtcaaaccaaaacagattgacgaaggcgcagaggttatcgtcctcttagatacttctcaaaaaatgactgaaaccgattttaatacggcaaaagaaaacatcaaaaaattagtgacaacactaacaggtacgacagataaagaaggaaagaatgtgtctcactataataatcgtaattcagttcgtttaattgacttttataggaaggtaggagaatctaccgatttatctggatgggatgccaaaaaaatcgatgaaaaacttaacgaagtttggaaaaaagctaaggatgactataatggatggggcgtagatttacagggtgccattcataaagcaagagaaatttttaatttagataaagaaaagaggtcgggtaaacgacaacatattgttttattttcccaaggagaatctacctttagttatgatattaaagataaatctaaaatggacaaagttgctgttgaggagcctgtgacttacagtaatccccttttcccttggcccttttactttgataccacaaccagaacacacaatgtggtgaatgatgcaaaaaaacttattgattttttaaataaattgggtatcagtcagtttaatggtgctgttgataacgttgctacggtaggaaatacccttttaggtctcggaagtttttttgggcttaagaatcctttggattatatttctttggcagatttagaaactagtaagttgaattccgaaaagtttgactattctagaagggtaggagaaggctataatttccgttcttattttgatagagaagttgataaggttggctttaaaaaaatcctagttgaaaaaatcaagggtaatctaaagaagttccaacctaaacaaacagatacctggttaagttctttgggattgaatagtatcaaagaaaaaatccaagattggatgattgataaagcgcttgataatctcttttatcgtcgtcagtaccaattctataaccacaatctctctgcccaagcagaagcgagaatggctagagaagaaggcataaaattttatgctgttgatgttactgaaccagagcgtattgcgaaagagattaattcccaaaaatatagtgaagcctatactaaccatctgaagaaaaaggctgaagaagctagagaacttgctaagaagcgtaatgagaagtttgataaatatctgaaagaaatgtctgaaagtcagaaattctttaaagacgttgaggatcctgagaaatttaaagatatcctaacagagcttaaagtgactgaaacctttgaggaaaaagtttcggttaataatagtgaacagcggaagagcaataaagaagttgaatataaaaaagcatcgtctaactcttcatttctttcattcattttctcaagttcaacaaatgaaagtataacttggacactttcaaaagataaactgcaaaaggctctacaatcaggggaaactttaaccttagagtataagttaaaaatccataaggacaaattcaagttagcgcctcaaacgagatcaaaacgttctctagatacctcagaaaacaaaaaatctgtaactgaaaaagtaataactagcgatgttaaatataagattaatgataaagaagtgaaaggtaaagaactagacgatgtctctttaacttacagtaaagaaaccgttcgtaagccacaggtggaaccaaatgttcctgatacacctcaggaaaaaccattgacaccgcttgcaccgtcagaaccttcacaaccatctattccagagacaccactgataccgtcagaaccttcagttccagagacatcaacaccagaaggtccaacagagggagaaaataatcttggtggtcagagtgaagagataacgattacagaagattctcaatcagggatgtctggtcaaaatcctggttctggaaatgaaacagtggttgaagacactcaaacaagtcaagaggatattgtacttggtggtccaggtcaagtgattgactttacagaagatagccaaccgggtatgtctggtaataatagccatactattacagaagattctaaaccaagtcaagaggatgaggtgataatcggcggtcaaggtcaggtgattgactttacagaagatactcaatctggtatgtctggggataatagccatacagatgggacagtgcttgaagaagactctaaaccaagtcaagaggatgaggtgataatcggcggtcaaggtcaagtgattgactttacagaagatacccaaaccggtatgtctggggctggacaagtagagagtccaacaatcaccgaagaaacccataaaccagaaataatcatgggcggtcaaagtgaccctattgatatggttgaggacactcttcctggtatgtctggctctaatgaagctactgttgtggaagaagacacacgtcctaaacttcaattccattttgataatgaagagcccgttcctgcaacggttccaaccgtttctcaaactcctattgctcaggtagaaagtaaagtgcctcatgccaaagcagagagtgcgttacctcaaactggagatacaaataaactagaaacgttctttaccattacagcactaactgttattggagcggcaggattactaggcaaaaaacgtcgtaataatcaaactgattaa

(19) United States
(12) Patent Application Publication
(10) Pub. No.: US 2004/0151737 A1
(43) Pub. Date: Aug. 5, 2004

(54) Streptococcal serum opacity factors and fibronectin-binding proteins and peptides thereof for the treatment and detection of streptococcal infection

(75) Inventor: Harry S. Courtney, West Memphis, AR (US)

Correspondence Address: SPECKMAN LAW GROUP PLLC, 1501 WESTERN AVE, SEATTLE, WA, 98101, US

(73) Assignee: University of Tennessee, Memphis, TN (US)

(21) Appl. No.: 10/771,931

(22) Filed: Feb. 4, 2004

Related U.S. Application Data
(60) Provisional application No. 60/446,061, filed on Feb. 5, 2003.

(51) Int. Cl. A61K 39/02

(52) U.S. Cl. 424/190.1

(57) ABSTRACT

Provided herein are S. pyogenes serum opacity factor (SOF)- and S. dysgalactiae fibronectin-binding protein-based polypeptide, polynucleotide, and antibody compositions and methods. Compositions provided herein are effective in eliciting opsonic and/or protective antibodies specific for S. pyogenes and/or S. dysgalactiae and, consequently, are useful inter alia for the treatment, diagnosis, and monitoring of streptococcal infections, including S. pyogenes and S. dysgalactiae infections, and diseases associated with S. pyogenes and S. dysgalactiae infections ranging from, but not limited to, mild and generally self-limiting infections of the pharynx and skin to more severe and life-threatening infections, such as toxic shock syndrome and necrotizing fasciitis. Compositions and methods provided herein will also find use in preventing, or minimizing the severity of, the major sequelae of streptococcal infections are acute rheumatic fever and acute glomerulonephritis, as well as associated autoimmune neurological disorders.

### REFERENCE TO PRIORITY APPLICATION

This application claims priority to U.S. Provisional Application No. 60/446,061 filed Feb. 5, 2003.

### BACKGROUND OF THE INVENTION

1. Technical Field of the Invention

The present invention relates generally to the fields of immunology and molecular biology. More specifically, this invention relates to Streptococcus pyogenes serum opacity factor (SOF)- and Streptococcal dysgalactiae (FnBA)-based antibody, polypeptide, and polynucleotide compositions, therapeutics and methods for the treatment and detection of streptococcal infection. Antibodies, polypeptides, and polynucleotides presented herein are useful, inter alia, as therapeutic agents effective in protecting against and/or in eliciting an opsonic and/or protective antibody response against streptococci such as the Group A Streptococcus pyogenes and Group C Streptococcus dysgalactiae. In addition, antibodies, polypeptides, and polynucleotides presented herein are also useful in diagnostic methods for the detection and monitoring of streptococcal infection.

2. Description of the Related Art

The Group A streptococcus, Streptococcus pyogenes, causes a variety of diseases ranging from mild and generally self-limiting infections of the pharynx and skin to more severe and life-threatening infections, such as toxic shock syndrome and necrotizing fasciitis. The major sequelae of Group A streptococcal infections are acute rheumatic fever and acute glomerulonephritis, which are thought to be due to autoimmune T- and B-cell responses induced by streptococcal products. Beachey et al., Vaccine 6:192-196 (1988); Cu et al., Kid. Int. 54:819-826 (1998); Cunningham, “Microorganisms and Autoimmune Disease,” pp. 13-66 (ed. Rose and Friedman, Plenum Publishing Corp., New York, 1996); Cunningham, Clin. Microbiol. Rev. 13:89-98 (2000); Dale et al., J. Exp. Med. 166:1825-1835 (1987); Dale et al., J. Exp. Med. 164:1785-1790 (1986); Fischetti, Clin. Microbiol. Rev. 2:285-314 (1989). Prior infections with Group A streptococci may also lead to autoimmune neurological disorders. Bodner, et al., Biol. Psychiatry 49: 807-810 (2001); Murphy et al., Arch. Pediatr. Adolesc. Med. 156:356-361 (2002); Swedo, Mol. Psychiatry 7:S24-25 (2002).

Serum opacity factor (SOF) is an ~100 kDa, surface-bound and released protein of Group A streptococci that causes opalescence of serum. Ward et al., Aust. J. Exp. Biol Med. Sci. 16:181-192 (1938); Krumwiede, J. Exp. Med. 100:629-638 (1954). It is composed of alternating variable and conserved domains and a highly conserved C-terminal domain with a LPXXG anchoring motif. Courtney et al., Mol. Microbiol. 32:89-98 (1999); Kreikemeyer et al., Mol. Microbiol. 17:137-145 (1995); Rakonjac et al., Infect. Immun. 63:622-631 (1995). The C-terminal domain contains a tandemly repeated peptide that binds fibronectin and fibrinogen. Courtney et al., Mol. Microbiol. 32:89-98 (1999); Courtney et al., Curr. Microbiol. 44:236-240 (2002); Kreikemeyer et al., Mol. Microbiol. 17:137-145 (1995); and Rakonjac et al., Infect. Immun. 63:622-631 (1995). The opacification of serum can be inhibited by antisera against type-specific determinants of SOF and this inhibition is the basis for the SOF typing scheme of Group A streptococci. Beall et al., Microbiol. 146:1195-1209 (2000).

SOF specifically cleaves the apolipoprotein A1 (Apo A1) in high-density lipoproteins (HDL). It has been suggested that the opalescence of serum resulted from the aggregation of HDL particles. Saravani et al., FEMS Microbiol Lett. 68:35-40 (1990). The sof gene from M type 22 Streptococcus pyogenes has been sequenced, and the deduced amino acid sequence was found to contain a fibronectin-binding domain separate and distinct from the domain for enzyme activity. Rakanjac et al., Infect. Immun. 63:622-631 (1995). Another sof gene from an unidentified serotype has also been cloned, sequenced and found to be almost identical to sof22. Kreikemeyer et al., Mol. Microbiol. 17:137-145 (1995).

Kreikemeyer found the sof gene in 43% of isolates from invasive Group A streptococcal infections and in 56% of streptococci isolated from wound, throat and skin infections. Strains of Group A streptococci that express SOF are also a common cause of impetigo and many are nephritogenic. Wannamaker, N. Engl. J. Med. 282:23-30 (1970); Bisno et al., N. Engl. J. Med. 334:240-245 (1996). The ability to opacify serum is also found in many strains of Group C streptococci as well as various staphylococci. The protein responsible for this opacity reaction has been identified in Group A streptococci, such as Streptococcus pyogenes, to be SOF while in Group C streptococci, such as Streptococcus dysgalactiae, the fibronectin-binding protein FnBA has been identified as an opacity factor. Courtney et al., Molecular Microbiology 32(1):89-98 (1999). In addition, Staphylococcus epidermidis produces a SOF and can cause infections of the respiratory tract and skin in humans.

SOF, in addition to M protein, is used to serotype Group A streptococci. This typing scheme is based on the observation that SOF contains type-specific determinants that co-vary with the type-specific determinants of M protein. Widdowson et al., J. Gen. Microbiol. 61:343-353 (1970) and Johnson et al., J. Med. Microbiol. 38:311-315 (1993). Thus, by determining the SOF type, the serotype can also be identified. Currently, there are more than 90 different M protein serotypes and ˜35% of these express SOF. Id.

The S. dysgalactiae fibronectin-binding protein FnBA has a significant degree of homology with SOF, consistent with its functioning as an opacity factor. Lindgren et al., Eur. J. Biochem. 214:819-827 (1993) and Courtney et al., Mol. Micro. 32(1):89-98 (1999). Based on the finding that two different streptococcal species express an opacity factor that bind fibronectin, it has been suggested that the linking of these two activities may be important to streptococcal virulence. Id.

SOF is not the only virulence factor in SOF-positive Group A streptococci. Both M-related proteins and M proteins are also required for virulence of M type 2 S. pyogenes. Podbielski et al., Mol. Microbiol. 19:429-441 (1996). Inactivation of mga in a SOF-positive, M type 49 strain resulted in a lack of expression of M49 protein, M-like protein, SOF49 and loss of virulence indicating that one or more of these proteins is required for virulence. McLandsborough et al., FEMS Microbiol. Lett. 128:45-51 (1995). Taken together, these data suggest that expression of SOF, M proteins and M-related proteins is required for full vurulence of SOF-positive streptococci. Although expression of the hyaluronate capsule is required for full virulence in SOF-negative streptococci, its role in the pathogenesis of infections due to SOF-positive streptococci has not been addressed. Wessels et al., Proc. Natl. Acad. Sci. USA 88:8317-8321 (1991).

Early efforts to develop a vaccine to prevent the diseases associated with streptoccal infections have, to date, focused on M proteins because infections in humans were found to elicit an immune response to M protein that was protective and long-lived. Lancefield, J. Exp. Med. 110:271-291 (1959). M proteins are the major virulence factor in Group A streptococci and confer the abilities to multiply in non-immune human blood and to attach to host cells. Courtney et al., Ann. Med. 34:77-87 (2002); Cunningham, Clin. Microbiol. Rev. 13:89-98 (2000); and Fischetti, Clin. Microbiol. Rev. 2:285-314 (1989). Structurally, M proteins are a-helical, coiled-coil proteins that radiate from the surface of the organism and are composed of a variable N-terminal half and a highly conserved C-terminal half. Id. The N-terminal 40-50 amino acids are hypervariable and elicit type-specific antisera.

Both the conserved and variable domains of M proteins are targets of current vaccine efforts and each approach has its own strengths and weaknesses. The major strength of a vaccine based on the conserved domains of M proteins is that protection is provided against both homologous and heterologous isotypes. Bessen et al., Infect. Immun. 56:2666-2672 (1988); Brandt et al., Infect. Immun. 68:6587-6594 (2000); Bronze et al., J. Immunol. 148:888-893 (1992); Olive et al., Infect. Immun. 70:2734-2738 (2002); Olive et al., Vaccine20:2816-2825 (2002); and Pruksakom et al., J. Immunol. 149:2729-2735 (1992). The major concern is that these conserved domains may stimulate T- and B-cell responses that target human tissues. Cunningham, “Microorganisms and Autoimmune Disease,” pp. 13-66 (ed. Rose and Friedman, Plenum Publishing Corp., New York, 1996); Dale et al., J. Exp. Med. 166:1825-1835 (1987); and Dale et al., J. Exp. Med. 164:1785-1790 (1986). Good and co-workers identified a peptide in the C-repeats of M proteins that elicits bactericidal antibodies that do not cross-react with human tissues. The level of bactericidal antibodies may not, however, be adequate in some cases. Olive et al., Infect. Immun. 70:2734-2738 (2002) and Olive et al., Vaccine 20:2816-2825 (2002).

The major strength of a vaccine based on the variable N-terminus is that a strong bactericidal antibody response is evoked and these antibodies are less likely to cross-react with human tissues. Dale et al., Vaccine 14:944-948 (1996); Hu et al., Infect. Immun. 70:2171-2177 (2002); U.S. Pat. Nos. 6,063,386 and 6,419,932; and U.S. patent application Publication No. 2002/0176863. The major problem is that protection is generally type-specific and there are more than 100 different M types of Group A streptococci. This problem has been addressed by developing multi-valent vaccines that target prevalent serotypes causing pharyngitis, invasive diseases, and rheumatic fever. Id. Thus, a 26-valent vaccine targeted 84% of all Group A streptococcal isolates and 74% of invasive isolates identified from 1998 to 2000. Id.

More recent investigations have identified a number of other vaccine candidates including the R28 protein (Stalhammar-Carlemalm et al., Mol. Microbiol. 33:208-219 (1999)); SPA (Dale et al., J. Clin. Invest. 103:1261-1268 (1999) and McLenan et al., Infect. Immun. 69:2943-2949 (2001)); C5a peptidase (Ji et al., Infect. Immun. 65:2080-2087 (1997)); the Group A carbohydrate (Salvadori et al., J. Infect. Dis. 171:593-600 (1995)); SFB1 (also termed protein F1; Guzman et al., J. Infect. Dis. 179:901-906 (1999); Medina et al., Eur. J. Immunol. 28: 1069-1077 (1998); and Schulze et al., Infect. Immun. 69:622-625 (2001)); FBP54 (Kawabata et al., Infect. Immun. 69:924-930 (2001)); and lipoteichoic acid (LTA, Dale et al., J. Infect. Dis. 169:319-323 (1994)). Some of these antigens will elicit protection against only a limited number of serotypes while other antigens, such as Group A carbohydrate, may require high concentrations of antibodies to be effective. Furthermore, the C5a peptidase, SFB1, and the R28 protein have not been shown to induce antibodies that opsonize Group A streptococci.

FBP54 evokes opsonic antibodies against two different serotypes, but its degree of coverage and efficacy of protection has not yet been thoroughly investigated. Kawabata et al., Infect. Immun. 69:924-930 (2001). LTA induced antibodies that blocked colonization but almost all Gram-positive bacteria produce LTA. Dale et al., J. Infect. Dis. 169:319-323 (1994). Therefore, a vaccine utilizing LTA would not be selective in the bacteria it targets. Because of these considerations, the M-protein-based vaccine is widely considered to be the most promising. However, not all types of M proteins evoke a protective antibody response and there are serotypes i n which a protective antigen (an antigen that evokes a protective immune response) has not yet been identified. Brandt et al., Infect. Immun. 68:6587-6594 (2000). Interestingly, the type-specific determinants of SOF usually co-vary with those of M proteins in a given strain and, thus, the M type can be predicted based on the SOF type. Id. Inactivation of SOF decreased the virulence of an M type 2 S. pyogenes in a mouse model indicating that it is a virulence determinant. Courtney et al., Mol. Microbiol. 32:89-98 (1999).

There remains a need in the art for improved methods and therapeutics for eliciting a protective immune response against a broad range of Streptococcus pyogenes infections.

### SUMMARY OF THE INVENTION

The present invention addresses these and other related needs by providing, inter alia, antibody, polypeptide, and polynucleotide based compositions and methods for the treatment, diagnosis and monitoring of streptococcal infection, more specifically, Streptococcus pyogenes and/or Streptococcus dysgalactiae infection. As noted above, antibodies, polypeptides, and polynucleotides presented herein are useful as therapeutic agents effective in protecting against and in eliciting an immune response that is protective against streptococcal infection. Inventive antibodies, polypeptides and polynucleotides are also useful in diagnostic methods for the detection and monitoring of a streptococcal infection, including Streptococcus pyogenes and/or Streptococcus dysgalactiae infection.

Thus, within certain aspects, the present invention provides isolated S. pyogenes serum opacity factor (SOF)-based polypeptides comprising one or more immunogenic portion(s) of an S. pyogenes SOF polypeptide. Within certain embodiments, S. pyogenes SOF-based polypeptides comprise one or more immunogenic portion(s) from one or more serum opacity factor(s) selected from the group consisting of SOF2 (SEQ ID NO: 1), SOF4 (SEQ ID NO: 3), and SOF28 (SEQ ID NO: 5). Within other embodiments, S. pyogenes SOF-based polypeptides comprise one or more immunogenic portion from one or more serum opacity factor isolated from an SOF-positive M type S. pyogenes strain wherein the serum opacity factor is selected from the group consisting of SOF 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

Within certain embodiments, immunogenic portions comprise at least 9 amino acids of an S. pyogenes serum opacity factor. Other embodiments provide SOF-based polypeptides that comprise at least 10, 11, 12, 13, 14, or 15 amino acids of an S. pyogenes serum opacity factor. Still further embodiments provide SOF-based polypeptides that comprise at least 16, 17, 18, 19, or 20 amino acids of an S. pyogenes serum opacity factor. Alternative embodiments provide SOF-based polypeptides that comprise at least 25, 30, 35, 40, 45, or 50 amino acids or at least 75, 100, 150, or 200 amino acids of an S. pyogenes serum opacity factor.

Exemplary SOF-based polypeptides of the present invention comprise one or more immunogenic epitope common to two or more S. pyogenes serotypes. Within such embodiments, common immunogenic SOF epitopes may be selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

Within other aspects, the present invention provides isolated S. dysgalactiae FnBA-based polypeptides comprising one or more immunogenic portion(s) of an S. dysgalactiae fibronectin binding protein polypeptide. Within certain embodiments, S. dysgalactiae FnBA-based polypeptides comprise one or more immunogenic portion(s) from FnBA (SEQ ID NO: 56).

Within certain embodiments, immunogenic portions comprise at least 9 amino acids of an S. dysgalactiae fibronectin-binding protein. Other embodiments provide fibronectin-binding protein-based polypeptides that comprise at least 10, 11, 12, 13, 14, or 15 amino acids of an S. dysgalactiae fibronectin-binding protein. Still further embodiments provide fibronectin-binding protein-based polypeptides that comprise at least 16, 17, 18, 19, or 20 amino acids of an S. dysgalactiae fibronectin-binding protein. Alternative embodiments provide fibronectin-binding protein-based polypeptides that comprise at least 25, 30, 35, 40, 45, or 50 amino acids or at least 75, 100, 150, or 200 amino acids of an S. dysgalactiae fibronectin-binding protein.

Other aspects of the present invention provide fusion proteins comprising two or more immunogenic portions of one or more S. pyogenes serum opacity factor polypeptide. Within such embodiments, fusion proteins may comprise two or more common immunogenic SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

Still further aspects of the present invention provide fusion proteins comprising one or more immunogenic portion of an S. pyogenes serum opacity factor polypeptide and one or more immunogenic portion of a non-SOF S. pyogenes polypeptide. Within such embodiments, fusion proteins may comprise one or more common immunogenic SOF epitope selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27) and an immunogenic portion of one or more non-SOF-based polypeptide selected from the group consisting of S. pyogenes M protein, R28 protein, SPA, C5a peptidase, SFB1 (also know as protein F1), and FBP54.

Yet additional aspects of the present invention provide fusion proteins comprising one or more immunogenic portion of an S. pyogenes serum opacity factor polypeptide and one or more immunogenic portion of a S. dysgalactiae fibronectin-binding polypeptide, such as FnBA. Within such embodiments, fusion proteins may comprise one or more common immunogenic SOF epitope selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27) and an immunogenic portion of an S. dysgalactiae fibronectin-binding polypeptide such as S. dysgalactiae FnBA polypeptide (SEQ ID NO: 56).

The present invention also provides cocktails comprising two or more immunogenic portions of an S. pyogenes SOF-based polypeptide as indicated above. Related aspects of the present invention provides cocktails comprising one or more immunogenic portion of an S. pyogenes SOF-based polypeptide and one or more immunogenic portion of an S. pyogenes non-SOF-based polypeptide. Exemplary suitable non-SOF-based polypeptides may be selected from the group consisting of S. pyogenes M protein, R28 protein, SPA, C5a peptidase, SFB1(also know as protein F1), and FBP54.

Other aspects of the present invention provide polynucleotides encoding each of the aforementioned polypeptides and fusion proteins.

Further aspects of the present invention provide antibodies and compositions comprising one or more antibody that specifically binds to an S. pyogenes serum opacity factor (SOF) and/or an S. dysgalactiae fibronectin-binding protein (FnBA). According to certain embodiments, SOF- and/or FnBA-specific antibodies are capable of facilitating opsonization of bacterium, including streptococci such as, for example S. pyogenes and S. dysgalactiae when the antibody is administered in vivo to a mammal, such as a human.

Within other embodiments, SOF-specific and/or FnBA-specific antibodies are capable of preventing adhesion of bacterium to the mucosal surfaces of a mammal, such as a human, thereby reducing bacterial colonization.

Antibodies according to the present invention may be either monoclonal antibodies or polyclonal antibodies. Within certain embodiments, antibodies are human monoclonal antibodies.

Other aspects of the present invention provide methods for eliciting an in vivo antibody response against a streptococcus, such as S. pyogenes and/or S. dysgalactiae, in a mammal. Such methods comprise the step of administering to the mammal a composition comprising an S. pyogenes SOF-based polypeptide and/or an S. dysgalactiae fibronectin-binding protein-based polypeptide. Within certain embodiments, the serum opacity factor (SOF)-based polypeptide comprises one or more immunogenic portion from one or more serum opacity factor selected from the group consisting of SOF2 (SEQ ID NO: 1), SOF4 (SEQ ID NO: 3), and SOF28 (SEQ ID NO: 5). Within still further embodiments, the serum opacity factor (SOF)-based polypeptide comprises one or more immunogenic portions from an S. pyogenes serotype selected from the group consisting of 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124. Within yet further embodiments, the S. dysgalactiae fibronectin-binding protein-based polypeptide is FnBA (SEQ ID NO: 56).

Within other aspects, such methods comprise a serum opacity factor (SOF)-based polypeptide comprising one or more common immunogenic epitope of an S. pyogenes SOF polypeptide selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

The present invention also provides methods for eliciting an in vivo antibody response against S. pyogenes in a mammal comprising the step of administering to the mammal a fusion protein comprising two or more immunogenic portions of one or more S. pyogenes serum opacity factor polypeptide. Within certain embodiments, the serum opacity factor is from an S. pyogenes wherein the serum opacity factor is selected from the group consisting of S. pyogenes SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107,109,110,112,113, 114,117,118, and 124.

In related embodiments, the present invention additionally provides methods for eliciting an in vivo antibody response against S. dysgalactiae in a mammal comprising the step of administering to the mammal a fusion protein comprising two or more immunogenic portions of one or more S. dysgalactiae fibrinogen-binding protein polypeptide. Within certain embodiments, the fibrinogen-binding protein is S. dysgalactiae FnBA (SEQ ID NO: 56).

Fusion proteins employed in methods of the present invention may comprise two or more common immunogenic SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

In other embodiments of the present methods, fusion proteins may comprise one or more immunogenic portions of an S. pyogenes serum opacity factor polypeptide and one or more immunogenic portions of a non-SOF S. pyogenes polypeptide wherein the non-SOF-based polypeptide may be selected from the group consisting of S. pyogenes M protein, S. pyogenes R28 protein, S. pyogenes SPA, S. pyogenes C5a peptidase, S. pyogenes SFB1 (also know as protein F1), S. pyogenes FBP54, and S. dysgalactiae FnBA.

Other aspects of the present invention provide methods for eliciting an in vivo antibody response against a streptococcus, such as, for example, S. pyogenes and S. dysgalactiae, in a mammal comprising the step of administering to the mammal one or more immunogenic portion of an S. pyogenes serum opacity factor polypeptide and/or one or more immunogenic portion of a non-SOF S. pyogenes polypeptide, such as S. dysgalactiae FnBA, as indicated herein above.

Still further aspects of the present invention provide methods for treating a streptococcal infection in a mammal, comprising the step of administering to the mammal an antibody that specifically binds to an S. pyogenes serum opacity factor and/or an S. dysgalactiae fibronectin-binding protein wherein the antibody is capable of facilitating opsonization of said streptococcus. By these methods, the S. pyogenes serum opacity factor may be selected from the group consisting of S. pyogenes SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124. The S. dysgalactiae fibronectin-binding protein may be selected from the group consisting of S. dysgalactiae FnBA (SEQ ID NO: 56).

Still further aspects of the present invention provide methods for detecting the presence of a streptococcus, such as, for example an S. pyogenes and/or an S. dysgalactiae, in a patient. By such methods, a biological sample, such as blood or serum, is obtained from the patient and tested for the presence of a streptococcal bacterium.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS AND SEQUENCE IDENTIFIERS

FIG. 1 is a graph depicting the cross-reaction of anti-SOF2 serum with SOF4 and SOF28. (See, Example 2). Microtiter wells were coated with SOF2 (circles), SOF4 (squares), or SOF28 (triangles). The coated wells were reacted with dilutions of rabbit preimmune serum (open circle) or rabbit anti-SOF2 serum (filled symbols). The reaction of preimmune serum with wells coated with SOF4 and SOF28 is not shown but was similar to that shown with SOF2.

FIG. 2 is a graph depicting bactericidal activity of anti-SOF2 serum. An inoculum of the indicated serotypes of S. pyogenes was mixed with rabbit anti-SOF2 serum or with preimmune serum, added to heparinized human blood, rotated for 3 hours at 37° C., and the numbers of CFU determined as described in Example 3. The means from 3 separate experiments ±SD are shown. M type 5 strain Manfredo is a SOF-negative strain and serves as a negative control.

FIG. 3 is a graph depicting the combined effects of anti-SOF2 serum and anti-M2 serum on opsonization of M type 2 S. pyogenes in human blood. Serial two-fold dilutions of rabbit anti-sM2(1-35) serum were added to an equal volume of NRS (closed circles) or anti-SOF2 (open circles). An inoculum of ~175 CFU and non-immune human blood were added. The mixtures were rotated for 3 hours, and the number of CFU determined as described in Example 4. When used alone without anti-sM2(1-35) serum, anti-SOF2 serum killed 33% of the streptococci. The concentration of anti-SOF2 serum used in this experiment was half of that used in the experiments depicted in the graph shown in FIG. 2.

FIG. 4 is a survival plot demonstrating that immunization of mice with SOF2 protects against challenge infections with SOF-positive Group A streptococci. (See, Example 7). Groups of five mice were immunized by IV injections of SOF2(38-1047) or SOF2(494-1047). Ten days later all ten immunized mice received an IP injection of SOF2(494-1047). At day 21 the immunized mice were challenged IP with ~5×10⁷ CFU of S. pyogenes, strain T2MR. Non-immunized control mice received an IP injection of ~5×10⁷ CFU. Both groups of mice that were immunized were combined since there was no difference in their rate of survival. The difference in survival between immunized and non-immunized mice was significant (Fisher's exact test, p=0.005).

FIG. 5 is a graph depicting antibody levels in mice immunized with SOF2ΔFBD. Ten mice were immunized with SOF2ΔFBD (closed circle) and nine mice were mock immunized (open circle) as described in Example 7. Serum was collected from the tail vein of each mouse, diluted 1:1000, and tested for reactivity with SOF2ΔFBD in ELISA assays. Each circle represents a single mouse.

FIG. 6 is a survival plot demonstrating that immunization of mice with SOF2ΔFBD protects against infections from SOF-positive Group A streptococci. Ten mice were subcutaneously immunized with SOF2ΔFBD and nine mice were mock immunized as described in Example 7. The mice were challenged by an IP injection of ~1×10⁷ CFU of S. pyogenes, strain T2MR, and the number of surviving mice was determined daily. The difference in survival between SOF2ΔFBD-immunized mice and mock-immunized mice was significant (Fisher's exact test, p=0.03).

FIG. 7 is a Western blot demonstrating cross-reactivity between SOF2 from S. pyogenes and FnBA from S. dysgalactiae.

SEQ ID NO: 1 is the amino acid sequence of S. pyogenes serum opacity factor (sof2) GenBank Accession No. AF019890.

SEQ ID NO: 2 is the nucleotide sequence of S. pyogenes serum opacity factor (sof2) GenBank Accession No. AF019890.

SEQ ID NO: 3 is the amino acid sequence of S. pyogenes serum opacity factor (sof4) GenBank Accession No. AY162273.

SEQ ID NO: 4 is the nucleotide sequence of S. pyogenes serum opacity factor (sof4) GenBank Accession No. AY162273.

SEQ ID NO: 5 is the amino acid sequence of S. pyogenes serum opacity factor (sof28) GenBank Accession No. AF082074.

SEQ ID NO: 6 is the nucleotide sequence of S. pyogenes serum opacity factor (sof28) GenBank Accession No. AF082074.

SEQ ID NO: 7 is the amino acid sequence of S. pyogenes serum opacity factor polypeptide SOF2-H(38-1047).

SEQ ID NO: 8 is the nucleotide sequence encoding S. pyogenes serum opacity factor polypeptide SOF2-H(38-1047) presented in SEQ ID NO: 7.

SEQ ID NO: 9 is the amino acid sequence of S. pyogenes serum opacity factor polypeptide SOF2-H(38-843).

SEQ ID NO: 10 is the nucleotide sequence encoding S. pyogenes serum opacity factor polypeptide SOF2-H(38-843) presented in SEQ ID NO: 9.

SEQ ID NO: 11 is the amino acid sequence of S. pyogenes serum opacity factor polypeptide SOF2-H(494-1047).

SEQ ID NO: 12 is the nucleotide sequence encoding S. pyogenes serum opacity factor polypeptide SOF2-H(494-1047) presented in SEQ ID NO: 11.

SEQ ID NO: 13 is the amino acid sequence of S. pyogenes serum opacity factor polypeptide SOF2-H(38-493).

SEQ ID NO: 14 is the nucleotide sequence encoding S. pyogenes serum opacity factor polypeptide SOF2-H(38-493) presented in SEQ ID NO: 13.

SEQ ID NO: 15 is the amino acid sequence of the following serum opacity factor (SOF) immunogenic portion: ETEPQTMDVEQYTVDKENS.

SEQ ID NO: 16 is the amino acid sequence of the following serum opacity factor (SOF) immunogenic portion: DIFDVKREVKTNGDGTLDVLT.

SEQ ID NO: 17 is the amino acid sequence of the following serum opacity factor (SOF) immunogenic portion: PKQIDEGADVMALLDVSQKM.

SEQ ID NO: 18 is the amino acid sequence of the following serum opacity factor (SOF) immunogenic portion: FDKAKEQIKKLVTTLT.

SEQ ID NO: 19 is the amino acid sequence of the following serum opacity factor (SOF) immunogenic portion: YNRRNSVRLMTFYR.

SEQ ID NO: 20 is the amino acid sequence of the following serum opacity factor (SOF) immunogenic portion: WGDVLQGAIHKAREIFNKEK.

SEQ ID NO: 21 is the amino acid sequence of the following serum opacity factor (SOF) immunogenic portion: RQHIVLFSQGESTFSYDIK.

SEQ ID NO: 22 is the amino acid sequence of the following serum opacity factor (SOF) immunogenic portion: TTSNPLFPWLPIFNHT.

SEQ ID NO: 23 is the amino acid sequence of the following serum opacity factor (SOF) immunogenic portion: FDYSKRVGEGYYYHSFSDR.

SEQ ID NO: 24 is the amino acid sequence of the following serum opacity factor (SOF) immunogenic portion: ERNEKFDNYLKEMSEGGK.

SEQ ID NO: 25 is the amino acid sequence of the following serum opacity factor (SOF) immunogenic portion: DVDKADKFKDTLTEL.

SEQ ID NO: 26 is the amino acid sequence of the following serum opacity factor (SOF) immunogenic portion: TKESLTWTISKD.

SEQ ID NO: 27 is the amino acid sequence of the following serum opacity factor (SOF) immunogenic portion: SLTLKYKLKVNKDKL.

SEQ ID NO: 28 is the amino acid sequence of serum opacity factor (SOF) fibrinogen-binding domain (FBD) (DITEDTQPGMSGSNDATVVEEDTAPQRPDVLVGGQSDPIDITED TQPGMSGSNDATVVEEDTVPKRPDILVGGQSDPIDITEDTQPGMSGSNDATVIEEDTK).

SEQ ID NO: 29 is the amino acid sequence of an exemplary SOF-based polypeptide comprising tandem repeats of a common immunogenic epitope (GASSVASSASSSSNGSVA SSSEPQMPQAQTAPQM).

SEQ ID NO: 30 is the partial amino acid sequence of S. pyogenes serum opacity factor (sof8) GenBank Accession No. AF138790.

SEQ ID NO: 31 is the partial amino acid sequence of S. pyogenes serum opacity factor (sof9) GenBank Accession No. AF174430.

SEQ ID NO: 32 is the partial amino acid sequence of S. pyogenes serum opacity factor (sof11) GenBank Accession No. AF141140.

SEQ ID NO: 33 is the partial amino acid sequence of S. pyogenes serum opacity factor (sof13) GenBank Accession No. AJ012314.

SEQ ID NO: 34 is the partial amino acid sequence of S. pyogenes serum opacity factor (sof22) GenBank Accession No. AF138791.

SEQ ID NO: 35 is the partial amino acid sequence of S. pyogenes serum opacity factor (sof25) GenBank Accession No. AF138795.

SEQ ID NO: 36 is the partial amino acid sequence of S. pyogenes serum opacity factor (sof27) GenBank Accession No. AF138796.

SEQ ID NO: 37 is the partial amino acid sequence of S. pyogenes serum opacity factor (sof44) GenBank Accession No. AF138798.

SEQ ID NO: 38 is the partial amino acid sequence of S. pyogenes serum opacity factor (sof48) GenBank Accession No. AF138799.

SEQ ID NO: 39 is the amino acid sequence of S. pyogenes serum opacity factor (sof49) GenBank Accession No. AF057697.

SEQ ID NO: 40 is the partial amino acid sequence of S. pyogenes serum opacity factor (sof58) GenBank Accession No. AF138801.

SEQ ID NO: 41 is the partial amino acid sequence of S. pyogenes serum opacity factor (sof59) GenBank Accession No. AF138802.

SEQ ID NO: 42 is the partial amino acid sequence of S. pyogenes serum opacity factor (sof60) GenBank Accession No. AF138803.

SEQ ID NO: 43 is the partial amino acid sequence of S. pyogenes serum opacity factor (sof61) GenBank Accession No. AF138804.

SEQ ID NO: 44 is the partial amino acid sequence of S. pyogenes serum opacity factor (sof62) GenBank Accession No. AF138805.

SEQ ID NO: 45 is the amino acid sequence of S. pyogenes serum opacity factor (sof63) GenBank Accession No. AF181974.

SEQ ID NO: 46 is the partial amino acid sequence of S. pyogenes serum opacity factor (sof66) GenBank Accession No. AF138807.

SEQ ID NO: 47 is the partial amino acid sequence of S. pyogenes serum opacity factor (sof68) GenBank Accession No. AF138808.

SEQ ID NO: 48 is the partial amino acid sequence of S. pyogenes serum opacity factor (sof73) GenBank Accession No. AF138809.

SEQ ID NO: 49 is the partial amino acid sequence of S. pyogenes serum opacity factor (sof75) GenBank Accession No. AF139736.

SEQ ID NO: 50 is the partial amino acid sequence of S. pyogenes serum opacity factor (sof76) GenBank Accession No. AF139734.

SEQ ID NO: 51 is the partial amino acid sequence of S. pyogenes serum opacity factor (sof77) GenBank Accession No. AF138810.

SEQ ID NO: 52 is the partial amino acid sequence of S. pyogenes serum opacity factor (sof78) GenBank Accession No. AF139739.

SEQ ID NO: 53 is the partial amino acid sequence of S. pyogenes strain SS1151 serum opacity factor (sof79) GenBank Accession No. AF192473.

SEQ ID NO: 54 is the partial amino acid sequence of S. pyogenes serum opacity factor (sof81) GenBank Accession No. AF138811.

SEQ ID NO: 55 is the partial amino acid sequence of S. pyogenes serum opacity factor (sof87) GenBank Accession No. AF139744.

SEQ ID NO: 56 is the amino acid sequence of S. dysgalactiae fibronectin-binding protein (FnBA) GenBank Accession No. CAA80121.

SEQ ID NO: 57 is the nucleotide sequence encoding the amino acid sequence of SEQ ID NO: 56 GenBank Accession No. Z22150.

### DETAILED DESCRIPTION OF THE INVENTION

The present invention is based on the observation that the Group A streptococcus Streptococcus pyogenes serum opacity factor (SOF) is capable of eliciting opsonic antibodies and/or a protective immune response against S. pyogenes infection. More specifically, as disclosed herein, it was found that in vivo administration of SOF and SOF-based polypeptides is effective in eliciting an antibody response against S. pyogenes in humans, rabbits, and mice. Furthermore, it was also found that antibodies raised against S. pyogenes SOF cross-react with a fibronectin-binding protein from S. dysgalactiae (i.e. FnBA). Thus, SOF and SOF-based polypeptides as well as FnBA and FnBA-based polypeptides according to the present invention will find utility in methods for the diagnosis and treatment of diseases caused by streptococcus, such as S. pyogenes and S. dysgalactiae, including, but not limited to, toxic shock syndrome, acute rheumatic fever and/or acute glomerulonephritis.

As used in this specification and the appended claims, the singular forms “a,” “an” and “the” include plural references unless the content clearly dictates otherwise.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al., “Molecular Cloning: A Laboratory Manual” (2nd Edition, 1989); Maniatis et al., “Molecular Cloning: A Laboratory Manual” (1982); “DNA Cloning: A Practical Approach, vol. I & II” (D. Glover, ed.); “Oligonucleotide Synthesis” (N. Gait, ed., 1984); “Nucleic Acid Hybridization” (B. Hames & S. Higgins, eds., 1985); “Transcription and Translation” (B. Hames & S. Higgins, eds., 1984); “Animal Cell Culture” (R. Freshney, ed., 1986); and Perbal, “A Practical Guide to Molecular Cloning” (1984). All publications, patents and patent applications cited herein, whether supra or infra, are hereby incorporated by reference in their entirety.

In general, polypeptides (including polypeptide fusion proteins and conjugates), polynucleotides and antibodies as described herein are isolated. An “isolated” polypeptide, polynucleotide, or antibody is one that is removed from its original environment. For example, an SOF-based or non-SOF-based polypeptide, fusion protein, or conjugate is “isolated” if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment. Antibodies are isolated if they are separated and/or fractionated from the blood, sera, ascites, culture media, or other fluid in which they are raised and/or expressed.

The compositions and methods of the present invention will be better understood through the detailed description of the following specific embodiments:

(a) Serum opacity factor (SOF)-based and/or fibronectin-binding protein (FnBA)-based polypeptide compositions effective in eliciting opsonic antibodies and/or a protective immune response against streptococcus, including, but not limited to S. pyogenes and S. dysgalactiae, and polynucleotides encoding polypeptides, including SOF-based polypeptides and FnBA-based polypeptides, effective in eliciting a protective immune response against streptococcus and vector systems for the expression of such polynucleotides;

(b) Compositions comprising one or more antibody directed against S. pyogenes SOF-based polypeptides and/or against S. dysgalactiae FnBA-based polypeptides;

(c) Methods for the treatment of streptococcal infection and associated diseases based on the in vivo administration to a mammal of an S. pyogenes SOF-based polypeptide, an S. dysgalactiae FnBA-based polypeptide, and/or antibodies raised against an S. pyogenes SOF-based polypeptide and/or an S. dysgalactiae FnBA-based polypeptide; and

(d) Methods for the diagnosis of streptococcal infection, such as S. pyogenes infection and/or S. dysgalactiae infection, and associated disease based on the detection of an S. pyogenes serum opacity factor and/or an S. dysgalactiae fibrinogen-binding protein.

Each of these embodiments is described in greater detail herein below.

### Streptococcus Pyogenes Serum Opacity Factor (SOF)- and Streptococcus Dysgalactiae (FnBA)-based Polypeptides, Fusion Proteins, and Complexes

Within certain embodiments, the present invention provides Streptococcus pyogenes serum opacity factor (SOF)-based polypeptides, Streptococcus dysgalactiae (FnBA)-based polypeptides, and compositions comprising SOF-based polypeptides and FnBA-based polypeptides that are effective in inducing an opsonic and/or protective immune response when administered in vivo to a mammal such as a mouse, rabbit, or human. Antibodies elicited by the in vivo administration of SOF-based polypeptides and/or FnBA-based polypeptides are capable of binding to one or more serotype of Group A and/or Group C streptococcus thereby facilitating bacterial opsonization and/or preventing or inhibiting the adhesion of bacteria to mucosal surfaces of mammals, including humans.

As used herein, the term “opsonization” refers to the process whereby bacteria, more specifically S. pyogenes or S. dysgalactiae, bind an antibody to its cell membranes thereby identifying the bacteria to the phagocytic system. Without wishing to be limited to a particular mode of action, it is believed that neutrophils and/or monocytes/macrophages can bind to the Fc portion of the bound anti-SOF and/or anti-FnBA antibody and phagocytize the bacteria that have been identified to them by the bound antibody. Alternatively or additionally, antibodies bound to the surface of S. pyogenes and/or S. dysgalactiae may undergo a conformational change that stimulates the deposition of complement C3 on the bacterial surface thereby facilitating neutrophil and/or monocyte/macrophage mediated phagocytosis through binding of the phagocytic cells to the bacteria through the cells' C3 receptors.

As used herein, the term “SOF-based polypeptide” is meant to include immunogenic portions of one or more SOF polypeptide of a SOF positive Group A streptococcus such as, for example, S. pyogenes. “SOF-based polypeptides” of the present invention are capable of eliciting an antibody response when administered in vivo to a mammal which antibodies are capable of facilitating bacterial opsonization when bound to a SOF polypeptide exposed on the surface of the bacteria. Alternatively or additionally, the antibodies elicited by in vivo administration of “SOF-based polypeptides” may prevent and/or inhibit adhesion of bacteria to the mucosal surfaces of the mammal.

Within certain embodiments, “SOF-based polypeptides” comprise one or more immunogenic portions comprising epitopes that are common to two or more S. pyogenes serotypes. Disclosed herein are “common epitopes” of SOF polypeptides exemplified by, but not limited to, the following amino acid sequences: ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

As used herein, the term “FnBA-based polypeptide” is meant to include immunogenic portions of one or more FnBA polypeptide of an FnBA positive Group C streptococcus such as, for example, S. dysgalactiae. “FnBA-based polypeptides” of the present invention are capable of eliciting an antibody response when administered in vivo to a mammal which antibodies are capable of facilitating bacterial opsonization when bound to a SOF polypeptide exposed on the surface of the bacteria. Alternatively or additionally, the antibodies elicited by in vivo administration of “FnBA-based polypeptides” may prevent and/or inhibit adhesion of bacteria to the mucosal surfaces of the mammal.

As used herein, the term “immunogenic” refers to the ability of a polypeptide, including a SOF-based polypeptide and/or a non-SOF-based polypeptide, such as an FnBA-based polypeptide, to elicit an immune response, preferably a protective antibody response, against streptococci. Within certain embodiments, antibodies raised against “immunogenic” SOF-based and/or a non-SOF-based polypeptides of the present invention are capable of facilitating opsonization of S. pyogenes and/or S. dysgalactiae when the antibody binds to the surface of the bacterium. Assay systems for determining the “immunogenic” properties of a candidate SOF-based and/or non-SOF-based polypeptide are presented herein in the Examples and references cited therein.

Exemplary suitable S. pyogenes SOF-based polypeptides comprise one or more immunogenic portion from one or more serum opacity factor from (1) the M type 2 S. pyogenes strain T2MR (SOF2, presented herein as SEQ ID NO: 1); (2) the M type 4 S. pyogenes strain 52936 (SOF4, presented herein as SEQ ID NO: 3); and/or (3) the M the 28 S. pyogenes strain 92448 (SOF28, presented herein as SEQ ID NO: 5). Polynucleotides encoding each of these S. pyogenes serum opacity factors are presented herein is SEQ ID NOs: 2, 4, and 6, respectively.

Equally suited to the practice of the present invention are SOF-based polypeptides comprising one or more immunogenic portions from one or more serum opacity factors from the following SOF-positive M type S. pyogenes strains: 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

Exemplary suitable S. dysgalactiae FnBA-based polypeptides comprise one or more immunogenic portion from one or more fibronectin-binding protein from S. dysgalactiae, including, but not limited to, FnBA (SEQ ID NO: 56).

As described in further detail herein, compositions of the present invention may comprise one or more SOF-based polypeptide and/or one or more FnBA-based polypeptide in the context of fusion proteins or in a cocktail. As used herein, the term “cocktail” refers to a mixture comprising one or more SOF-based polypeptide and/or one or more FnBA-based polypeptide wherein individual polypeptides are not complexed one to the other through a covalent bond, such as a peptide bond. Within certain embodiments, fusion proteins and/or cocktails may contain two or more SOF-based polypeptides, two or more FnBA-based polypeptides, and/or may contain one or more additional non-SOF-based polypeptide.

As used herein, the term “non-SOF-based polypeptide” includes an immunogenic portion from a second S. pyogenes polypeptide including, but not limited to, S. pyogenes M protein, R28 protein, SPA, C5a peptidase, SFB1 (also know as protein F1), FBP54, and/or S. dysgalactiae FnBA. Within certain preferred embodiments, fusion proteins and/or cocktails may comprise two or more immunogenic portions of an S. pyogenes M protein as disclosed in U.S. Pat. Nos. 6,063,386 and 6,419,932 and U.S. Patent Application Publication No. 2002/0176863, each of which is hereby incorporated by reference in its entirety. Other embodiments of the present invention provide fusion proteins and/or cocktails comprising one or more SOF-based polypeptide and the 26-valent M protein based polypeptide presented in Hu et al., Infect. Immun. 70:2171-2177 (2002), incorporated herein by reference in its entirety.

A non-SOF-based polypeptide fusion partner may, for example, stimulate a protective immune response, preferably an antibody response, against S. pyogenes and/or may assist in expressing the protein (an expression enhancer) at higher yields than the native recombinant SOF-based polypeptide. Certain preferred fusion partners are both immunogenic and expression enhancing. Other fusion partners may be selected so as to increase the solubility of the protein or to enable the protein to be targeted to desired intracellular compartments. Still further fusion partners include affinity tags, which facilitate purification of the protein.

SOF-based polypeptides suited for use in fusion proteins and/or cocktails of the present invention comprise an immunogenic portion of a SOF polypeptide. For example, SOF-based polypeptides may comprise at least 9 amino acids of an S. pyogenes serum opacity factor such as SOF2, SOF4, or SOF28 as depicted in SEQ ID NOs: 1, 3, or 5, respectively. Certain embodiments comprise SOF-based polypeptides that comprise at least 10, 11, 12, 13, 14, or 15 amino acids of an S. pyogenes serum opacity factor as depicted in SEQ ID NO: 1, 3, and/or 5. Still further embodiments comprise SOF-based polypeptides that comprise at least 16, 17, 18, 19, or 20 amino acids of an S. pyogenes serum opacity factor as depicted in SEQ ID NO: 1, 3, and/or 5. Alternative embodiments comprise SOF-based polypeptides that comprise at least 25, 30, 35, 40, 45, or 50 amino acids or at least 75, 100, 150, or 200 amino acids of an S. pyogenes serum opacity factor as depicted in SEQ ID NO: 1, 3, and/or 5.

Within certain embodiments, SOF-based polypeptides suitable for use in the fusion proteins and/or cocktails of the present invention are the SOF2-based polypeptides SOF2-H(38-1047), SOF2-H(38-843), SOF2-H(494-1047), and SOF-H(38-493), disclosed herein a SEQ ID NOs 7, 9, 11, and 13, respectively. These exemplary SOF-based polypeptides are encoded by the polynucleotides disclosed herein as SEQ ID NOs 8, 10, 12, and 14, respectively.

Alternatively or additionally, fusion proteins and/or cocktails comprising SOF-based polypeptides may comprise common immunogenic epitopes of two or more S. pyogenes SOF polypeptides as exemplified by the immunogenic portions presented herein as SEQ ID NOs: 15-27.

Within certain embodiments, fusion proteins may employ an N-terminal moiety and a C-terminal moiety wherein the N-terminal moiety includes at least an immunogenic portion of one or more S. pyogenes serum opacity factor and the C-terminal moiety may include at least a portion of a second streptococcal polypeptide. Exemplary suitable serum opacity factors are SOF2, SOF4, and SOF28 from S. pyogenes strains T2MR, 5 2936, and 9 2448, respectively, presented herein in SEQ ID NO: 1, 3, and/or 5. Equally preferred are fusion proteins wherein either the C-terminal moiety or the N-terminal moiety includes at least an immunogenic portion of one or more non-SOF-based polypeptide such as, for example, an S. pyogenes M protein, R28 protein, SPA, C5a peptidase, SFB1 (also know as protein F1), and/or FBP54.

Thus, “SOF-based polypeptide fusion proteins,” as disclosed herein, include covalent complexes formed between, at a minimum, the N-terminal moiety and the C-terminal moiety. For example, SOF-based polypeptide fusion proteins may comprise an N- or C-terminal moiety including at least about 9 amino acids of one or more immunogenic SOF-based polypeptide and a C- or N-terminal moiety including at least a portion of a second non-SOF-based S. pyogenes polypeptide. Other embodiments provide fusion proteins comprising at least 10, 11, 12, 13, 14, or 15 amino acids of one or more SOF-based polypeptide. Still further embodiments provide fusion proteins comprising at least 16, 17, 18, 19, or 20 amino acids of one or more SOF-based polypeptide. Alternative embodiments provide fusion proteins comprising at least 25, 30, 35, 40, 45, or 50 amino acids of one or more SOF-based polypeptide or comprising at least 75, 100, 150, or 200 amino acids of one or more SOF-based polypeptide.

It will be understood that SOF-based polypeptide fusion proteins may comprise more than two SOF-based polypeptides. For examples, SOF-based polypeptide fusion proteins may comprise 3, 4, 5, 6, 7, 8, 9, or 10 SOF-based polypeptides. Other embodiments provide SOF-based polypeptide fusion proteins comprising at least about 15, 20, or 25 SOF-based polypeptides. SOF-based polypeptide fusion proteins may also comprise one or more non-SOF-based polypeptide. For examples, SOF-based polypeptide fusion proteins may comprise 3, 4, 5, 6, 7, 8, 9, or 10 non-SOF-based polypeptides. Other embodiments provide SOF-based polypeptide fusion proteins comprising at least about 15, 20, or 25 non-SOF-based polypeptides.

Within other embodiments, fusion proteins may employ an N-terminal moiety and a C-terminal moiety wherein the N-terminal moiety includes at least an immunogenic portion of one or more S. dysgalactiae fibronectin-binding protein and the C-terminal moiety may include at least a portion of a second streptococcal polypeptide. An exemplary suitable fibronectin-binding protein is FnBA from S. dysgalactiae presented herein in SEQ ID NO: 56. Equally preferred are fusion proteins wherein either the C-terminal moiety or the N-terminal moiety includes at least an immunogenic portion of one or more non-SOF-based polypeptide such as, for example, an S. pyogenes M protein, R28 protein, SPA, C5a peptidase, SFB1 (also know as protein F1), and/or FBP54.

SOF-based and non-SOF-based polypeptides and fusion proteins according to the present invention may be synthesized by conventional polypeptide synthesis methodology. For example, polypeptides and fusion proteins may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146 (1963). Equipment for automated synthesis of polypeptides is commercially available from suppliers such as Perkin Elmer/Applied BioSystems Division (Foster City, Calif.), and may be operated according to the manufacturer's instructions.

Alternatively, conventional molecular biology and recombinant DNA methodology may be employed to generate polynucleotides encoding SOF-based and non-SOF-based polypeptides and fusion proteins. Such methodologies are explained fully in the literature. See, e.g., Sambrook et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1 984); Animal Cell Culture (R. Freshney, ed., 1986); Perbal, A Practical Guide to Molecular Cloning (1984). Each of these publications is incorporated by reference in their entirety.

Briefly, DNA sequences encoding SOF-based and non-SOF-based polypeptides may be ligated into an appropriate expression vector wherein the expression vector comprises a transcriptional promoter in operable linkage to the polynucleotide encoding the polypeptide and transcription termination signals 3′ to the polynucleotide encoding the polypeptide. Suitable expression vectors may also provide translational start sites, Kozak sequences to direct translation initiation, and stop codons to end translation. In addition, preferred expression vectors may also comprise one or more polynucleotide sequences that encode polypeptides, such as His-His-His-His-His-His or the FLAG® sequence A sp-Tyr-Lys-Asp-Asp-Asp-Asp-Lys (Sigma-Aldrich, St. Louis, Mo.), which facilitates affinity purification of the SOF-based polypeptide.

Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. Supernatants from suitable host/vector systems which secrete the recombinant polypeptide and/or fusion protein into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify the recombinant polypeptide and/or fusion protein.

In addition to the SOF-based fusion proteins, which are generated by expression of DNA constructs, it will be appreciated that two or more SOF-based polypeptides or one or more SOF-based and one or more non-SOF-based polypeptide may be coupled one to the other through chemical means, such as by conventional coupling techniques. Methodologies for generating such polypeptide complexes are well known and readily available in the art. For example, two or more such polypeptide moieties may be coupled using a dehydrating agent such as dicyclohexylcarbodiimide (DCCI) to form a peptide bond between the two peptides. Alternatively, linkages may be formed through sulfhydryl groups, epsilon amino groups, carboxyl groups or other reactive groups present in the polypeptides, using commercially available reagents. (Pierce Co., Rockford, Ill.).

Conventional molecular biology and recombinant DNA techniques for generating fusion proteins are explained fully in the literature and are available by reference to the methodologies disclosed herein above for recombinant methodologies for the generation of SOF-based and FnBA-based polypeptides. Briefly, polynucleotide sequences encoding the SOF-based and FnBA-based polypeptide moieties may be assembled separately, and ligated into an appropriate expression vector. The 3′ end of the polynucleotide encoding the N-terminal moiety is ligated, with or without a peptide linker, to the 5′ end of the polynucleotide encoding the C-terminal moiety so that the reading frames of the sequences are in phase. This permits translation into a single fusion protein that retains the biological activity of both component polypeptides.

A peptide linker sequence may be employed to separate individual polypeptide moieties by a distance sufficient to ensure that each polypeptide properly folds into its native secondary and tertiary structures. Such a peptide linker sequence may be incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional amino acids on the SOF-based and/or FnBA-based polypeptides; and (3) the lack of hydrophobic or charged residues that might react with functional amino acids on the SOF-based and/or FnBA-based polypeptide.

Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46 (1985); Murphy et al., Proc. Natl. Acad. Sci. USA 83:8258-8262 (1986); U.S. Pat. No. 4,935,233; and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

It will be appreciated that SOF-based and/or FnBA-based polypeptides and fusion proteins according to the present invention encompass fragments, derivatives, and variants thereof so long as the fragments, derivatives, and variants do not substantially affect the functional properties of the SOF-based and/or FnBA-based polypeptides and fusion proteins. Thus, equally suited to the practice of the present invention are SOF-based and FnBA-based polypeptides and fusion proteins comprising sequence variations within the amino acid sequences of one or more of the SOF-based and/or FnBA-based polypeptide moieties. For example, the present invention contemplates SOF-based polypeptides and fusion proteins wherein one or more polypeptide moiety is at least 70% identical with an immunogenic portion of any of the SOF2, SOF4, and/or SOF28 amino acid sequences recited in SEQ ID NOs: 1, 3, and/or 5, respectively. More preferred are polypeptide moieties that are at least 80%, 90%, 95% and 98% identical to immunogenic portions of any of the SOF2, SOF4, and/or SOF28 amino acid sequences recited in SEQ ID NOs: 1, 3, and/or 5, respectively.

Exemplary FnBA-based polypeptides and fusion proteins comprise one or more polypeptide moiety is at least 70% identical with an immunogenic portion of FnBA (SEQ ID NO: 56). More preferred are polypeptide moieties that are at least 80%, 90%, 95% and 98% identical to immunogenic portions of FnBA (SEQ ID NO: 56).

A polypeptide or protein “fragment, derivative, and variant,” as used herein, is a polypeptide or protein that differs from a native polypeptide or protein in one or more substitutions, deletions, additions and/or insertions, such that the functional activity of the polypeptide or protein is not substantially diminished. In other words, the ability of a variant to specifically elicit a protective antibody response may be enhanced or unchanged, relative to the SOF-based and/or FnBA-based polypeptide or fusion protein, or may be diminished by less that 50%, and preferably less than 20%, relative to the native protein, without affecting the efficacy of the resulting SOF-based and/or FnBA-based polypeptide or fusion protein. Generally, suitable SOF-based and/or FnBA-based polypeptide variants may be characterized by assessing the ability to elicit a SOF-specific antibody response.

As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 70%, more preferably at least 80% or at least 90%, more preferably yet at least 95%, and most preferably, at least 98% identity to a sequence of the present invention. The percentage identity is determined by aligning the two sequences to be compared as described below, determining the number of identical residues in the aligned portion, dividing that number by the total number of residues in the inventive (queried) sequence, and multiplying the result by 100. In addition to exhibiting the recited level of sequence similarity, variant sequences of the present invention preferably exhibit a functionality that is substantially similar to the functionality of the sequence against which the variant is compared.

Variants may contain “conservative amino acid substitutions,” defined as a substitution in which one amino acid is substituted for another amino acid that has similar properties, such that the secondary structure and hydropathic nature of the polypeptide is substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes.

Functional fragments, derivatives, and variants of a polypeptide may be identified by first preparing fragments of the polypeptide by either chemical or enzymatic digestion of the polypeptide, or by mutation analysis of the polynucleotide that encodes the polypeptide and subsequent expression of the resulting mutant polypeptides. The polypeptide fragments or mutant polypeptides are then tested to determine which portions retain biological activity, using, for example, the representative assays provided below.

Fragments, derivatives, and variants of the inventive polypeptides may also be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized the Merrifield solid-phase synthesis method as discussed above.

Variants may also be prepared using standard mutagenesis techniques, such as oligonucleotide-directed, site-specific mutagenesis. Kunkel, Proc. Natl. Acad. Sci. USA 82:488-492 (1985). Sections of polynucleotide sequence may also be removed using standard techniques to permit preparation of truncated polypeptides. Variants may additionally, or alternatively, be modified by, for example, the deletion or addition of amino acids that have minimal influence on the immunogenicity, secondary structure and hydropathic nature of the polypeptide.

Polypeptide fragments, derivatives, and variants preferably exhibit at least about 70%, more preferably at least about 80% or 90% and most preferably at least about 95% or 98% sequence identity to the native polypeptide or protein. Polypeptide sequences may be aligned, and percentages of identical amino acids in a specified region may be determined against another polypeptide, using computer algorithms that are publicly available. The alignment and identity of polypeptide sequences may be examined using the BLASTP algorithm. The BLASTP algorithm is described in Pearson and Lipman, Proc. Natl. Acad. Sci. USA 85:2444-2448 (1988); and in Pearson, Methods in Enzymol. 183:63-98 (1990).

The BLASTP software is available on the NCBI anonymous FTP server and is available from the National Center for Biotechnology Information (NCBI), National Library of Medicine, Building 38A, Room 8N805, Bethesda, Md. 20894. The BLASTP algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTP, is described at NCBI's website and in the publication of Altschul et al., “Gapped BLAST and PSI-BLAST: a new generation of protein database search programs,” Nucleic Acids Res. 25:3389-3402 (1997).

The “hits” to one or more database sequences by a queried sequence produced by BLASTP, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The percentage identity of a polypeptide sequence is determined by aligning polypeptide sequences using appropriate algorithms, such as BLASTP, set to default parameters; identifying the number of identical amino acids over the aligned portions; dividing the number of identical amino acids by the total number of amino acids of the polypeptide of the present invention; and then multiplying by 100 to determine the percentage identity.

The BLASTP algorithm also produces “Expect” values for polypeptide alignments. The Expect value (E) indicates the number of hits one can “expect” to see over a certain number of contiguous sequences by chance when searching a database of a certain size. The Expect value is used as a significance threshold for determining whether the hit to a database indicates true similarity. For example, an E value of 0.1 assigned to a polypeptide hit is interpreted as meaning that in a database of the size of the SwissProt database, one might expect to see 0.1 matches over the aligned portion of the sequence with a similar score simply by chance. By this criterion, the aligned and matched portions of the sequences then have a probability of 90% of being related. For sequences having an E value of 0.01 or less over aligned and matched portions, the probability of finding a match by chance in the SwissProt database is 1% or less using the BLASTP algorithm.

According to one embodiment, “variant” SOF polypeptides and/or FnBA polypeptides, with reference to each of polypeptides of the present invention, preferably comprise sequences having the same number or fewer amino acids than each of the SOF polypeptides and/or FnBA polypeptides of the present invention and producing an E value of 0.01 or less when compared to the polypeptide of the present invention.

In addition to having a specified percentage identity to an inventive polypeptide, fusion protein, variant polypeptides preferably have additional structure and/or functional features in common with the inventive polypeptide. Polypeptides having a specified degree of identity to an SOF-based and/or FnBA-based polypeptide of the present invention share a high degree of similarity in their primary structure and have substantially similar functional properties. In addition to sharing a high degree of similarity in their primary structure to polypeptides of the present invention, polypeptides having a specified degree of identity to an inventive polypeptide preferably have at least one of the following features: (i) they have substantially the same functional properties as an inventive SOF-based and/or FnBA-based polypeptide; or (ii) they contain identifiable domains in common.

Polypeptides and fusion proteins of the present invention may further comprise a carrier moiety linked to the SOF-based or FnBA-based polypeptide or fusion protein. Within certain embodiments, the polypeptide and/or fusion protein is linked to the carrier moiety by an amino acid linker. Generally, carrier moieties are advantageously employed to enhance the immunogenicity of the polypeptide and/or fusion protein.

Antibodies Directed Against S. Pyogenes Serum Opacity Factor and S. Dysgalacitae Fibronectin-binding Domain Polypeptides

As noted above, the present invention also provides antibodies, and antigen-binding fragments thereof, that specifically bind to an S. pyogenes serum opacity factor and/or an S. dysgalactiae fibronectin-binding protein including, but not limited to, SOF2, SOF4, and SOF28, from S. pyogenes strains T2MR, 52936, and 92448, respectively, and FnBA from S. dysgalactiae, presented herein in SEQ ID NOs 1, 3, 5, and 56 respectively. Preferred antibodies are protective against streptococcal infection, including S. pyogenes and/or S. dysgalactiae infection and, within certain embodiments, are capable of binding to S. pyogenes and/or S. dysgalactiae, thereby, facilitating bacterial opsonization. Inventive antibodies are effective in reducing the number of streptococcal bacteria in a mammal when the antibodies are administered in vivo to the mammal.

As used herein, the term “antibody” includes and is most preferably an immunoglobulin or functional equivalent or fragment thereof. Thus, the term “antibody” includes parts, fragments, precursor forms, derivatives, variants, and genetically engineered or naturally mutated forms thereof and included amino acid substitutions and labeling with chemicals and/or radioisotopes and the like, so long as the resulting derivative and/or variant retains at least a substantial amount or antigen binding specificity and/or affinity.

Preferred mammalian antibodies are human antibodies, including monoclonal antibodies. As used herein, the term “antibody” broadly includes both antibody heavy and light chains as well as all isotypes of antibodies, including IgA, IgD, IgE, IgG1, IgG2a, IgG2b, IgM, and also encompasses antigen binding fragments thereof, including, but not limited to, Fab, F(ab′)₂, Fc, and scFv.

An antibody, or antigen-binding fragment thereof, is said to “specifically bind” to an 5. pyogenes serum opacity factor and/or an S. dysgalactiae fibronectin-binding protein if it reacts at a detectable level (within, for example, an ELISA) with a SOF and/or an FnBA polypeptide, and does not react detectably with unrelated proteins under similar conditions. As used herein, “binding” refers to a noncovalent association between two separate molecules such that a complex is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant may be determined using methods well known in the art.

Anti-SOF and anti-FnBA antibodies, and binding fragments thereof, may be further capable of differentiating between patients with and without a streptococcal infection using the representative assays provided herein. In other words, antibodies or other binding agents that bind to an SOF polypeptide and/or an FnBA polypeptide will generate a signal indicating the presence of streptococcus in at least about 20% of infected patients, and will generate a negative signal indicating the absence of infection in at least about 90% of individuals without infection. To determine whether a binding agent satisfies this requirement, biological samples (e.g., blood and/or sera) from patients with and without a streptococcal infection (as determined using standard clinical tests) may be assayed as described herein for the presence of SOF polypeptides and/or FnBA polypeptides that bind to the antibody or antigen binding fragment thereof. It will be apparent that a statistically significant number of samples with and without infection should be assayed. Each antibody should satisfy the above criteria; however, those of ordinary skill in the art will recognize that antibodies may be used in combination to improve sensitivity.

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogenic portion of an SOF polypeptide and/or an FnBA polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogenic SOF and/or FnBA polypeptide is injected into the animal host, preferably according to a predetermined schedule incorporating one or more booster immunizations, and the animals are bled periodically. Polyclonal antibodies specific for the SOF polypeptide may then be purified from such antisera by, for example, affinity chromatography using the polypeptide coupled to a suitable solid support.

Monoclonal antibodies specific for an immunogenic SOF and/or FnBA polypeptide may be prepared, for example, using the technique of Kohler and Milstein, Eur. J. Immunol. 6:511-519 (1976), and improvements thereto. Briefly, these methods involve the preparation of immortal cell lines capable of producing antibodies having the desired specificity (i.e. reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab, Fc, and scFv fragments, which may be prepared using standard techniques. Briefly, immunoglobulins may be purified from serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

### Therapeutic Compositions and Methods Employing S. pyogenes Serum Opacity Factor or S. Dysgalactiae Fibronectin-binding Protein Polypeptides, Polynucleotides, and Antibodies

Polypeptides, polynucleotides, and antibodies of the present invention are useful as therapeutic agents for the treatment of Group A and/or Group C streptococcal infections such as, for example, S. pyogenes and S. dysgalactiae infection. Thus, the present invention provides compositions comprising one or more SOF-based and/or FnBA-based polypeptide, polynucleotide, or antibody, as described herein above, and a biologically acceptable diluent or adjuvant. Compositions comprising one or more polypeptide and/or polynucleotide are suitable for eliciting opsonic and/or protective antibodies to S. pyogenes and/or S. dysgalactiae as discussed herein above.

Appropriate biologically acceptable diluents or adjuvants for the present compositions may be selected from a wide range of diluent or adjuvants as readily known to one of skill in the art, as is the development of suitable dosing and treatment regimens for using the particular compositions described herein in a variety of treatment regimens, including, for example, oral, parenteral, intravenous, intranasal, and intramuscular administration. It will be evident that the precise dose of the polypeptide, polynucleotide, and/or antibody compositions will vary depending upon the precise polypeptide, polynucleotide, and/or antibody used and the corresponding rate of clearance.

Exemplary diluents include phosphate-buffered saline. Particularly preferred is a dose of the therapeutic composition suspending in 25 ml of PBS, pH 7.2, containing 5 mg/ml kanamycin sulfate and I mg/ml each of paraaminobenzoic acid (PABA) and 2, 3-dihydrobenzoic acid (DHB).

Thus, within certain embodiments, the present invention provides methods for eliciting an in vivo antibody response against streptococcus in a mammal. Exemplary methods comprise the step of administering to the mammal a composition comprising an S. pyogenes SOF-based polypeptide. Within certain embodiments, the serum opacity factor (SOF)-based polypeptide comprises one or more immunogenic portions from one or more serum opacity factor selected from the group consisting of SOF2 (SEQ ID NO: 1), SOF4 (SEQ ID NO: 3), and S OF28 (SEQ ID NO: 5). Within still further embodiments, the S. pyogenes serum opacity factor is selected from the group consisting of SOF 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124. Within other aspects, the serum opacity factor (SOF)-based polypeptide comprises one or more common immunogenic epitope of an S. pyogenes SOF polypeptide selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

Alternative exemplary methods comprise the step of administering to the mammal a composition comprising an S. dysgalactiae FnBA-based polypeptide. Within certain embodiments, the FnBA-based polypeptide comprises one or more immunogenic portions from the FnBA depicted in SEQ ID NO: 56.

The present invention also provides methods for eliciting an in vivo antibody response against streptococcus in a mammal comprising the step of administering to the mammal a fusion protein comprising two or more immunogenic portions of one or more S. pyogenes serum opacity factor polypeptide and/or one or more S. dysgalactiae fibronectin-binding domain polypeptide. Within certain embodiments, the serum opacity factor is from an S. pyogenes selected from the group consisting of S. pyogenes M types 2 ( SEQ ID N 0: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124. Within other embodiments, the S. dysgalactiae fibronectin-binding protein is FnBA (SEQ ID NO: 56). Fusion proteins that are suitable in the methods of the present invention are described in further detail herein above.

Still further aspects of the present invention provide methods for treating a streptococcal infection in a mammal, comprising the step of administering to the mammal an antibody that specifically binds to an S. pyogenes serum opacity factor and/or an S. dysgalactiae fibronectin-binding protein wherein the antibody is capable of facilitating opsonization of said streptococcus. By these methods, the S. pyogenes serum opacity factor may be selected from the group consisting of S. pyogenes SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114,

117, 118, and 124. The S. dysgalactiae fibronectin-binding protein may be FnBA (SEQ ID NO: 56). Fusion proteins that are suitable in the methods of the present invention are described in further detail herein above.

Antibodies that are suitable in the methods of the present invention are described in further detail herein above.

In certain embodiments, the therapeutic compositions, fusion proteins, and/or antibodies disclosed herein may be delivered via oral administration to a mammal. As such, these compositions may be formulated with an inert diluent or with an edible carrier, or they may be enclosed in hard- or soft-shell gelatin capsule, or they may be compressed into tablets.

The compositions, fusion proteins, and/or antibodies of the present invention may also be administered parenterally. Mammals, in particular humans, immunized parenterally with a sufficient amount of the therapeutic polypeptide and/or polynucleotide composition of the present invention develop opsonic and/or protective antibodies directed to the epitopes of the immunogenic polypeptides. Non-limiting examples of such parenteral routes of administration are intracutaneous and intramuscular.

Compositions, fusion proteins, and/or antibodies may also be administered intranasally. For intranasal administration, a mammal may receive between about 50 μg to about 10 mg of purified antigen in an appropriate diluent for administration. An intranasal treatment regimen may be particularly well suited when the vaccine is constructed to evoke secretory or mucosal immunity since nasopharyngeal infection is a common route of infection in humans.

In accordance with the invention, the therapeutic composition, fusion proteins, and/or antibodies may be administered singly in series or advantageously in a mixture or cocktail of multiple compositions to elicit broad spectrum immunity against multiple S. pyogenes and/or S. dysgalactiae serotypes.

### Diagnostic Methods Employing S. Pyogenes Serum Opacity Factor and/or S. Dysgalactiae Fibronectin-binding Protein Polypeptides, Polynucleotides, and Antibodies

Polypeptides, polynucleotides, and/or antibodies of the present invention are useful as diagnostic agents in methods for the detection and monitoring of streptococcal infection, including, but not limited to S. pyogenes and/or S. dysgalactiae infection. In general, streptococcal infections may be detected in a patient based on the presence of a serum opacity factor and/or a fibronectin-binding protein or polynucleotides encoding a serum opacity factor and/or a fibronectin-binding protein in a biological sample such as, for example, blood and serum obtained from the patient. For example, the antibodies, or fragments thereof, disclosed herein may permit the detection of the level of SOF in the biological sample. Alternatively, polynucleotide primers or probes may be used to detect the level of mRNA encoding a polypeptide, which is correlative of the extent of streptococcal infection.

There are a variety of assay formats known to those of ordinary skill in the art for using antibodies to detect polypeptide markers in a sample. See, e.g., Harlow and Lane, Antibodies, supra. In general, the presence or absence of an S. pyogenes and/or S. dysgalactiae infection in a patient may be determined by (a) contacting a biological sample obtained from a patient with an antibody, of fragment thereof; (b) detecting in the sample a level of polypeptide that binds to the antibody; and (c) comparing the level of polypeptide with a predetermined cut-off value.

Within certain embodiments, the assay involves the use of a first antibody immobilized on a solid support to bind to and remove the polypeptide from the remainder of the sample. The bound SOF and/or FnBA polypeptide may then be detected using a second antibody comprising an attached reporter group or label where the second antibody specifically binds to the first antibody/SOF and/or FnBA polypeptide complex. Generally, the reporter group or label is a radionuclide or fluorescent molecule.

Alternatively, a competitive assay may be utilized, in which an isolated or purified SOF and/or FnBA polypeptide is labeled with a reporter group and allowed to bind to an antibody immobilized on a solid support following incubation of the immobilized antibody with a biological sample to be tested for the presence of an SOF and/or FnBA polypeptide. The extent to which components of the biological sample inhibit the binding of the labeled polypeptide to the immobilized antibody is indicative of the presence of an SOF and/or FnBA polypeptide in the biological sample. Suitable SOF polypeptides for use within such assays include SOF2, SOF4, and/or SOF28, from S. pyogenes strains T2MR, 52936, and 92448, respectively, disclosed herein in SEQ ID NOs 1, 3, and 5, respectively, or immunogenic portions thereof. A suitable FnBA polypeptide for use within such assays is FnBA from S. dysgalactiae, disclosed herein in SEQ ID NO: 56.

Suitable solid support that may be employed in the methods of the present invention may be any material known to those of ordinary skill in the art to which a polypeptide and/or antibody may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The antibody may be immobilized on the solid support using a variety of techniques known to those of skill in the art, which are described in the patent and scientific literature. In the context of the present invention, the term “immobilized” refers to both noncovalent association, such as adsorption, and covalent attachment (which may be a direct linkage between an antibody and functional groups on the support or may be a linkage by way of a cross-linking agent).

Immobilization by adsorption to a well in a microtiter plate or to a membrane is generally preferred. In such cases, adsorption may be achieved by contacting the antibody, of functional fragment thereof, in a suitable buffer, with the solid support for a suitable amount of time. The contact time caries with temperature, but is typically between about 1 hour and about 1 day. In general, contacting a well of a plastic microtiter plate (such as polystyrene or polyvinylchloride) with an amount of antibody ranging from about 10 ng to about 10 μg and preferably about 100 ng to about 1 μg is sufficient to immobilize an adequate amount of the antibody for use in a detection method.

As noted above, the present invention also provided polynucleotide-based methods for detecting, in a biological sample, a Group A streptococci such as, for example, S. pyogenes and/or a Group C streptococci such as, for example, S. dysgalactiae. Exemplary methods described herein are based upon the hybridization and/or amplification of a polynucleotide encoding a SOF and/or FnBA polypeptide and the detection of the hybridized and/or amplified polynucleotide.

Thus, the SOF polynucleotides encoding S. pyogenes SOF2, SOF4, and SOF28 presented herein as SEQ ID NOs 2, 4, and 6, respectively, and the FnBA polynucleotide encoding S. dysgalactiae presented herein as SEQ ID NO: 57 can be advantageously used as probes or primers for polynucleotide hybridization and amplification. As such, it is contemplated that polynucleotide segments that comprise a sequence region of at least about a 15 nucleotide long contiguous sequence that has the same sequence as, or is complementary to, a 15 nucleotide lone contiguous sequence that has the same sequence as, or is complementary to, a 15 nucleotide lone contiguous sequence disclosed herein will find particular utility. Longer contiguous identical or complementary sequence, e.g., those of about 20, 30, 40, 50, 100, 200, 500, 1000 (including all intermediate lengths) and even up to full-length sequences will also be of use in certain embodiments of the present invention.

The following Examples are offered by way of illustration not limitation.

### EXAMPLES

### Example 1

### Organisms and Growth Conditions

The SOF-positive strains of Streptococcus pyogenes used in this study were the M type 2 strain T2MR, the M type 4 strain 52936, and the M type 28 strain 92448. The M type 5 strain Manfredo is SOF-negative. The organisms were grown in Todd-Hewitt broth supplemented with 1.5% yeast extract (THY) at 37° C.

### Example 2

### Anti-SOF2 Antiserum Cross-reacts with SOF Polypeptides from Various S. pyogenes Serotypes

This Example demonstrates that antisera containing antibodies raised against SOF, SOF peptides, and anti-SOF cross-reacted with SOF polypeptides from various S. pyogenes serotypes.

The sof2, sof4, and sof28 genes from strains T2MR, 52936, and 92448, respectively, were amplified by PCR, ligated into the pTric His vector, introduced into E. coli Top10, expressed as histidine fusion proteins, and purified by metal-affinity chromatography as previously described. Courtney et al., Mol. Microbiol. 32:89-98 (1999). SOF2-H(38-1047) (SEQ ID NO: 7), SOF2-H(494-1047) (SEQ ID NO: 11), and SOF2-H(38-843) (SEQ ID NO: 9) are truncated forms of SOF2 spanning the indicated amino acid residues and were constructed and purified as previously described. Id. Herein, SOF2-H(38-843) is also referred to as SOF2ΔFBD to emphasize that the fibronectin-binding domain was deleted.

Rabbit antiserum against SOF2-H(38-1047) was prepared as previously described. Id. The sequences of sof2 and sof28 were previously published and correspond to GenBank Accession Nos. AF019890 and AF082074, respectively. Id. The sof4 gene was ligated into pCRII, was sequenced using M13 forward and reverse primers, and was assigned GenBank Accession No. AY162273.

The binding specificity of the anti-SOF2-H(38-1047) antiserum was tested in enzyme-linked immunosorbant (ELISA) assays. Wells of a micro titer plate were coated with purified recombinant SOF2, SOF4, and SOF28 (10 μg/ml in 0.01 M sodium bicarbonate, pH 9.5). Control wells were coated with bovine serum albumin (BSA). After coating, all wells were blocked with BSA, 1 mg/ml in PBS. Serial 1:2 dilutions of a 1:1,000 dilution of rabbit anti-SOF2-H(38-1047) or preimmune serum were added to the wells and incubated for 30 min at 37° C. The wells were washed, and a 1:2,000 dilution of peroxidase-labeled goat anti-rabbit immunoglobulins was added. After 30 min, the wells were washed and the substrate tetramethybenzidine was added. After color development, the absorbance at 650 nm was measured. The average value of wells coated with BSA served as a blank and was subtracted from all other values. All samples were tested in duplicate.

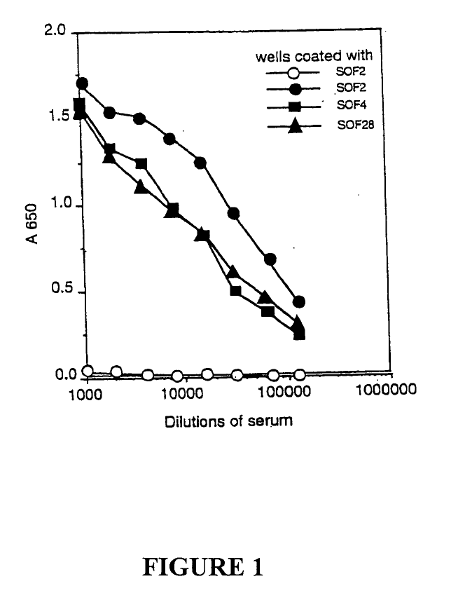

Rabbit antiserum against SOF2-H(38-1047) gave a strong reaction with SOF2 in ELISA assays as exhibited by a positive signal at a 1:128,000 dilution (FIG. 1). The anti-SOF2-H(38-1047) antiserum also strongly cross-reacted with SOF4 and SOF28. Because there is ˜60% homology between SOF2 and SOF28 and ˜53% homology between SOF2 and SOF4, this degree of cross-reactivity demonstrated that a significant proportion of the antibodies were directed against common SOF epitopes.

### Example 3

### Bactericidal Activity of Anti-SOF2 Antiserum

Streptococci were grown in THY to an O.D. of ~0.08 at 530 nm and diluted 1:10,000. 20 μl of this dilution were added to a tube containing 200 μl of anti-SOF2 serum or preimmune serum and 400 μl of heparinized human blood from a non-immune donor. The blood was rotated for 3 hours at 37° C. and the number of CFU was determined by plating dilutions on blood agar plates. The bactericidal assays were repeated on three separate occasions. In assays testing the combined effects of anti-sM2(1-35) serum and anti-SOF2 serum, 100 μl of the serial 1:2 dilutions of anti-sM2(1-35) were added to 100 μl of anti-SOF2 or normal rabbit serum (NRS). The mixtures were added to 400 μl of heparinized human blood and treated as described above. The percentage of Streptococci killed in the bactericidal assays was calculated by the formula: percent killing=[1-(number of CFU in anti-SOF2 serum/number of CFU in preimmune serum)]×100.

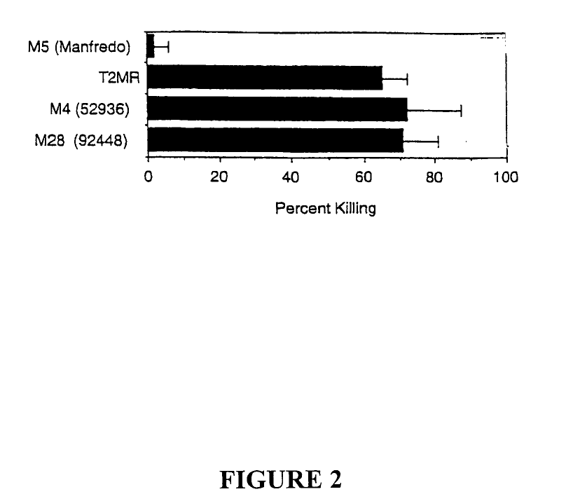

The ability of the rabbit antisera to SOF2 to opsonize M types 2, 4, and 28 of S. pyogenes was tested in non-immune human blood (FIG. 2). Rabbit antisera to SOF2-H(38-1047) not only opsonized and killed M type 2 S. pyogenes (65% killing), but also opsonized M types 4 and 28 (72% and 71% killing respectively). Two separate control experiments were performed to ensure that the antiserum did not aggregate the Streptococci. In one experiment, an identical inoculum was added to pre-immune serum and to anti-SOF2 serum, the mixtures were shaken, and the number of CFU determined by plating.

There was no difference in the numbers of CFU in the inocula, indicating that no aggregation occurred due to anti-SOF serum. In a second experiment, Streptococci were added to freshly prepared human plasma containing either preimmune serum or anti-SOF2 serum. After 3 hours of rotation, the numbers of CFU were determined. Again, no significant difference in the number of CFU was found between preimmune and immune serum indicating that anti-SOF2 serum did not aggregate the Streptococci. The results of the second experiment also demonstrated that neutrophils were needed in order to kill the Streptococci and that antibodies and complement alone were insufficient.

Next, it was of interest to determine if humans also produce opsonic antibodies to SOF2 (see, Table 1). A donor was selected whose serum inhibited the serum opacity reaction of SOF2. The antibodies to SOF2 were purified from this serum by affinity chromatography utilizing either SOF2-H(38-1047) or SOF2ΔFBD as the matrix and tested in bactericidal assays. Affinity purified antibodies were mixed with the indicated number of CFU of S. pyogenes, strain T2MR, and added to human blood as described herein. The number of CFU after 3 hours of rotation was determined by plating dilutions of the mixtures. Controls consisted of adding human IgG equivalent to the amount of affinity-purified SOF antibodies except for experiment 1 where Tris-saline buffer was used.

In two separate experiments, antibodies eluted from SOF2ΔFBD killed 40% and 43% of Streptococci in a bactericidal assay in whole human blood. Antibodies eluted from SOF2-H(38-1047) killed 73% of the Streptococci. These results indicate that SOF stimulates the production of bactericidal antibodies in humans. Although not conclusive, the data suggest that the fibronectin-binding domain of SOF contributes to this response.

TABLE 1
Opsonization of M Type 2 S. pyogenes by Affinity-purified
Human Antibodies to SOF2

| | Experiment 1 | Experiment 2 | Experiment 3 |
|---|---|---|---|
| Affinity Matrix | SOF2ΔFBD | SOF2ΔFBD | SOF2-H(38-1047) |
| | (SEQ ID NO: 9) | (SEQ ID NO: 9) | (SEQ ID NO: 7) |
| Inoculum, CFU | 72 | 40 | 85 |
| CFU in control | 51,600 | 44,800 | 54,720 |
| buffer/IgG | | | |
| CFU in | 31,200 | 25,600 | 14,760 |
| purified | | | |
| antibodies | | | |
| Percent killing | 40 | 43 | 73 |

### Bactericidal Effect of Combining Anti-M2 and Anti-SOF2 Antisera

To prepare anti sM2(1-35) antisera, the first 35 amino acids of the mature M2 protein were synthesized with a C-terminal cysteine residue used to cross-link the peptide to keyhole limpet hemocyanin (KLH) as previously described. Bronze et al., J. Immunol. 148:888-893 (1992). The conjugated peptide (500 μg/ml) was emulsified in complete Freund's adjuvant (CFA) and injected subcutaneously into New Zealand white rabbits. Booster injections of 500 μg in phosphate buffered saline (PBS) were given at 4, 8, 10, and 15 weeks.

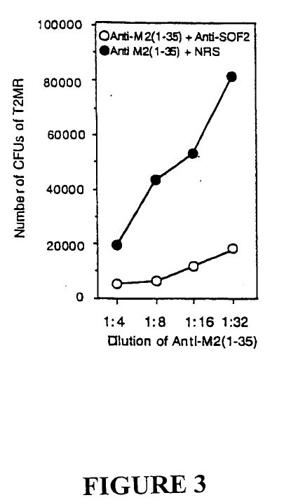

To further evaluate the potential of SOF as a vaccine candidate, the ability of anti-SOF2 serum to enhance the opsonic effect of antiserum to M protein was assessed. Serial 1:2 dilutions of rabbit antisera against a synthetic peptide copying the first 35 amino acids from the N-terminus of M protein from type 2 S. pyogenes, anti-sM2(1-35), were added to normal rabbit serum or to rabbit anti-SOF2 serum. S. pyogenes, strain T2MR, and non-immune human blood were added, and the mixtures treated as described in the bactericidal assays. Antiserum to SOF dramatically enhanced the ability of antisera against the M2 protein to opsonize and kill Group A Streptococci (FIG. 3).

### Example 5

### The Serum Opacity Reaction and its Inhibition

The ability of SOF in the culture supernatant of Streptococci to opacify serum was tested by centrifugation of overnight cultures of the organisms, sterilization of the media by filtration, and addition of 100 μl of the filtrate to one ml of horse serum. After incubation at 37° C. for three hours, the absorbance at 405 nm was recorded. Assays for neutralization of the opacity reaction consisted of preincubating 100 μl of neutralizing serum and 100 μl of culture supernatant for 30 min at 37° C., then adding 1 ml of horse serum, and recording the absorbance at 405 nm after 3 hours and after an overnight incubation. In some cases, purified recombinant SOF (1 μg/ml) was used instead of culture supernatants in the inhibition experiments described above.

### Example 6

### Purification of Human Anti-SOF Antibodies

A human donor was selected whose serum inhibited the serum opacity reaction of SOF2. The donor's serum was first chromatographed over a QAE-Sephadex column to remove other serum proteins that might have bound to SOF. The QAE flow through containing the antibodies was then added to a column of SOF2-H(38-1047) or SOF2ΔFBD covalently linked to agarose. The columns were washed with buffer, and bound proteins were eluted with 0.05 M sodium acetate, 0.1 M glycine, pH 3.0. The pH of the eluate was immediately neutralized by dialysis against PBS. The eluted antibodies retained their ability to inhibit the serum opacity reaction of SOF2.

### Example 7

### Mouse Toxicity and Protection Assays

Five NIH Swiss mice received IV injections in the tail vein of 100 μg of SOF2-H(38-1047) in 0.1 ml of PBS and 5 mice were injected IV with 100 μg of SOF2-H(494-1047) in 0.1 ml of PBS. The mice were evaluated daily for signs of toxicity such as ruffled fur, lethargy, weight loss, abnormal movements, or death. After 10 days, all ten mice received an intraperitoneal (IP) booster injection of 100 μg of SOF2-H(494-1047). At day 21, mice were challenged with ˜5×10⁷ CFU of T2MR by IP injection and the number of deaths recorded daily. As a control, 15 non-immunized mice were injected IP with ˜5×10⁷CFU of T2MR.

A second mouse protection study was undertaken to determine the effectiveness of SOF2ΔFBD immunizations in protecting mice against challenge infections. Ten NIH Swiss mice were injected subcutaneously with 25 μg of SOF2ΔFBD in CFA. Nine control mice received a subcutaneous injection of CFA. After two weeks, the mice were boosted with an intramuscular (IM) injection of 25 μg of SOF2ΔFBD in PBS. Control mice received PBS injections. Two weeks after the booster injections, all mice were challenged by an IP injection of ˜1×10⁷ CFU of T2MR. The number of surviving mice was recorded daily. Moribund mice were sacrificed and recorded as a death.

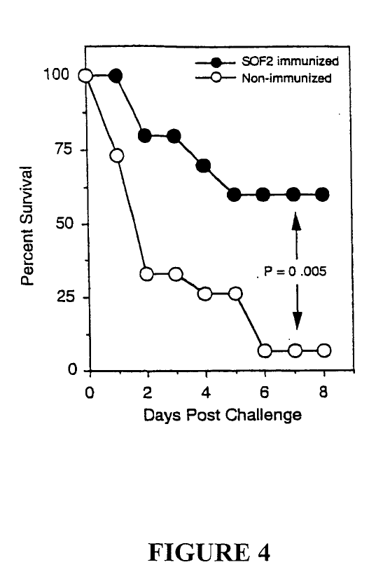

This experiment was initially designed to determine if SOF was toxic to mice. Five mice were injected IV with 100 μg of SOF2-H(38-1047) and 5 mice were injected IV with 100 μg of SOF2-H(494-1047). SOF2-H(38-1047) encompasses the mature SOF2 protein and opacifies serum. SOF2-H(494-1047) does not opacify serum and served as a negative control. None of the mice exhibited any visible signs of illness, indicating that SOF2 is not overtly toxic to mice under these conditions. The mice were then used to determine if vaccination against SOF2 would protect against Group A streptococcal infections. The mice were boosted by an IP injection of SOF2-H(494-1047) and challenged IP with ~5×10⁷ CFU of M type 2 strain T2MR 11 days later. As a negative control, 15 non-immunized mice were also challenged IP with T2MR. Only 4 of the 10 mice immunized with SOF2 died, whereas 14 of the 15 mice that were not immunized died (FIG. 4). These results demonstrated that immunization with SOF2 protected mice against infections from SOF-positive Group A Streptococci.

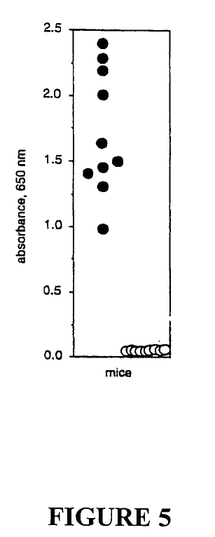

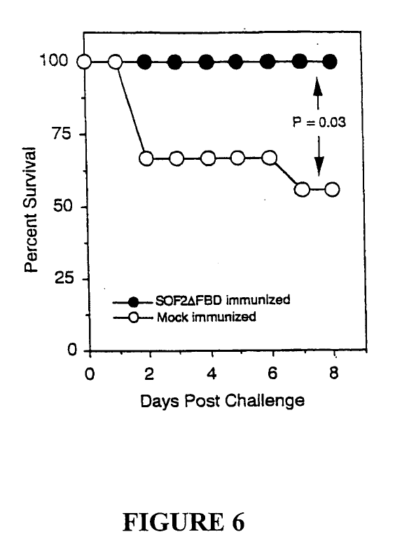

Next, we wanted to determine if the fibronectin-binding domain of SOF was required to induce protection in mice. Ten mice were immunized with SOF2ΔFBD in CFA, and 9 mice were mock immunized with CFA. After a booster injection, blood was obtained from the tail vein of mice and tested for antibodies to SOF. The immunized mice developed significant levels of antibodies to SOF2ΔFBD, whereas the mock-immunized mice did not (FIG. 5). All of the mice were challenged with ~1×10⁷ CFU of T2MR and the number of surviving mice was monitored daily. None of the immunized mice died, whereas 4 of the 9 mock-immunized mice died (FIG. 6). These data provide additional evidence that SOF induces a protective immune response and that the fibronectin-binding domain of SOF is not required for this response.

### Example 8

### Expression of an S. Pyogenes Serum Opacity Factor Fibronectin-binding Domain, Generation of Rabbit Polyclonal Antisera Thereto, and Assay for Bacteriocidal Activity

This Example discloses the expression of a S. pyogenes SOF fibrinogen-binding domain (FBD), the generation of rabbit polyclonal antisera specific for the SOF FBD, and demonstration of the bacteriocidal activity of the rabbit antisera.

The following peptide comprises the fibronectin-binding domain of S. pyogenes serum opacity factor: DITEDTQPGMSGSNDATVVEEDTAPQRPDVLVGGQSDPIDITEDTQPGM SGSNDATVVEEDTVPKRPDILVGGQSDPIDITEDTQPGMSGSNDATVIEEDTK (SEQ ID NO: 28). The DNA sequence that encodes this region is amplified by PCR and ligated into a pTrcHis plasmid that encodes a histidine tag at the N-terminus of the expressed protein. The protein is purified by Metal affinity chromatography. The purified protein is emulsified in complete Freund's adjuvant and injected subcutaneously into rabbits. The sera from rabbits is tested in the bactericidal assay as described in Examples 3 and 4.

### Example 9

### Repeat Sequences within Various SOF Polypeptides

Several short peptides are repeated three or more times within the various serotypes of SOF proteins. These common repeated epitopes candidates for SOF-based polypeptide therapeutics. The sequence of DNA that encodes these proteins is synthesized in tandem and in frame such that a single recombinant protein is expressed. An exemplary SOF-based polypeptide is presented herein as SEQ ID NO: 29 (GASSVASSASSSSNGSVASSSEP QMPQAQTAPQM). The synthesized DNA is incorporated into a pTrcHis vector, transformed into E. coli, and the recombinant protein purified by metal affinity chromatography. The purified protein is cross-linked to keyhole limpet hemocyanin (KLH) and injected subcutaneously into rabbits. The rabbit polyclonal antiserum is tested in standard bactericidal assays as described herein in Examples 3 and 4. Orientation of each of the short sequences is tested for capability to elicit opsonic antibodies against S. pyogenes.

### Example 10

### Anti-S. pyogenes SOF2 Polyclonal Antisera Cross-reacts with FnBA from S. dysgalactiae

This Example demonstrates that antisera raised against S. pyogenes SOF2 cross-reacts with FnBA from S. dysgalactiae.

The gene for S. dysgalactiae FnBA was amplified by PCR, ligated into a pBAD vector (Invitrogen Corp.; Carlsbad, Calif.), and transformed into E. coli. As a control, the pBAD vector without insert was transformed into E. coli. E. coli with vector only or E. coli expressing Fnba were lysed with SDS and the lysates electrophoresed on a polyacrylamide gel under reducing conditions. The proteins in the gel were electrophorectically transferred to nitrocellulose. The nitrocellulose was then blocked with bovine serum albumin and then reacted with a 1:1000 dilution of rabbit antiserum against recombinant S OF2 followed by reaction with a 1:1000 dilution of peroxidase-labeled goat anti-rabbit IgG (Boehringer-Mannheim; Indianapolis, Ind.). The substrate 4-chloro-1-naphtol (Sigma-Aldrich; St. Louis, Mo.) was added. After color development the nitrocellulose was washed with buffer.

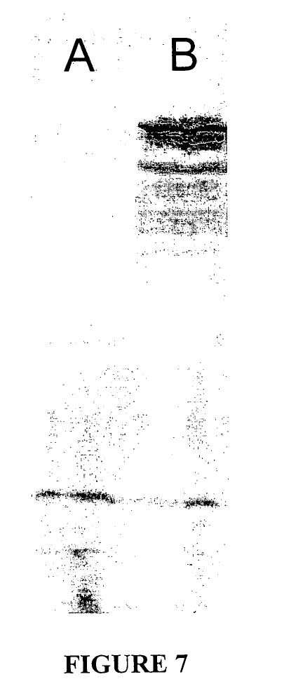

The Western blot demonstrating c ross-reactivity between SOF2 and FnBA from S. dysgalactiae is presented in FIG. 7. The reaction of anti-SOF2 serum with FnBA in lane B indicates that FnBA and SOF2 have shared epitopes.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

What is claimed is:

1. A composition comprising one or more immunogenic portions from one or more Group A streptococci serum opacity factor(s) (SOF) and a biologically acceptable diluent wherein said polypeptide is capable of eliciting a protective immune response when administered in vivo to a mammal.

2. The composition of claim 1 wherein said Group A streptococci is Streptococcus pyogenes.

3. The composition of claim 2 wherein said SOF is selected from the group consisting of S. pyogenes SOF2 (SEQ ID NO: 1), SOF4 (SEQID NO: 3), and SOF28 (SEQ ID NO: 5).

4. The composition of claim 2 wherein said SOF is selected from the group consisting of S. pyogenes SOF 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

5. A composition comprising one or more immunogenic portions from one or more Group C streptococci fibronectin-binding protein (FnBA) and a biologically acceptable diluent wherein said polypeptide is capable of eliciting a protective immune response when administered in vivo to a mammal.

6. The composition of claim 5 wherein said Group C streptococci is Streptococcus dysgalactiae.

7. The composition of claim 6 wherein said FnBA is selected from the group consisting of S. dysgalactiae FnBA.

8. A composition comprising one or more common immunogenic S. pyogenes SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27) and a biologically acceptable diluent or adjuvant.

9. A fusion protein comprising two or more immunogenic portions of one or more S. pyogenes serum opacity factor polypeptide wherein said S. pyogenes serum opacity factor is selected from the group consisting of SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

10. The fusion protein of claim 9 wherein said fusion protein comprises two or more common immunogenic SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

11. A fusion protein comprising one or more immunogenic portions of an S. pyogenes serum opacity factor polypeptide and one or more immunogenic portions of a non-SOF S. pyogenes polypeptide.

12. The fusion protein of claim 11 wherein said immunogenic portions of an S. pyogenes serum opacity factor polypeptide comprises one or more common immunogenic S. pyogenes SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

13. The fusion protein of claim 11 wherein said non-SOF-based polypeptide is selected from the group consisting of S. pyogenes M protein, R28 protein, SPA, C5a peptidase, SFB1 (also know as protein F1), and FBP54.

14. A cocktail comprising two or more immunogenic portions of a two or more S. pyogenes serum opacity factor polypeptides wherein said S. pyogenes serum opacity factor is selected from the group consisting of SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

15. The cocktail of claim 14 wherein one or more of said serum opacity factor polypeptides comprises a common immunogenic SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

16. A cocktail comprising one or more immunogenic portion of an S. pyogenes serum opacity factor polypeptide and one or more immunogenic portion of a non-SOF S. pyogenes polypeptide.

17. The cocktail of claim 16 wherein said cocktail comprises one or more common immunogenic S. pyogenes SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

18. The cocktail of claim 16 wherein said non-SOF-based polypeptide is selected from the group consisting of S. pyogenes M protein, R28 protein, SPA, C5a peptidase, SFB1 (also know as protein F1), and FBP54.

19. An isolated antibody that specifically binds to an S. pyogenes serum opacity factor wherein said antibody is capable of facilitating opsonization of said S. pyogenes.

20. The antibody of claim 19 wherein said S. pyogenes serum opacity factor is selected from the group consisting of S. pyogenes SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

21. The antibody of claim 19 wherein said serum opacity factor is selected from the group consisting of SOF2 (SEQ ID NO: 1), SOF4 (SEQ ID NO: 3), and SOF28 (SEQ ID NO: 5).

22. The antibody of claim 15 wherein said serum opacity factor polypeptides comprises a common immunogenic SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

23. A method for eliciting an in vivo antibody response against S. pyogenes in a mammal, said method comprising the step of administering to said mammal a composition comprising a S. pyogenes SOF-based polypeptide.

24. The method of claim 23 wherein said serum opacity factor (SOF)-based polypeptide comprises one or more immunogenic portions from one or more serum opacity factor selected from the group consisting of SOF2 (SEQ ID NO: 1), SOF4 (SEQ ID NO: 3), and SOF28 (SEQ ID NO: 5).

25. The method of claim 23 wherein said serum opacity factor (SOF)-based polypeptide comprises one or more common immunogenic epitope of an S. pyogenes SOF polypeptide selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

26. The method of claim 23 wherein said S. pyogenes serum opacity factor is selected from the group consisting of SOF 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

27. A method of eliciting an in vivo antibody response against S. pyogenes in a mammal, said method comprising the step of administering to said mammal a fusion protein comprising two or more immunogenic portions of one or more S. pyogenes serum opacity factor polypeptides wherein said S. pyogenes serum opacity factor is selected from the group consisting of S. pyogenes SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

28. The method of claim 27 wherein said fusion protein comprises two or more common immunogenic SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

29. The method of claim 27 wherein said fusion protein comprises one or more immunogenic portions of an S. pyogenes serum opacity factor polypeptide and one or more immunogenic portions of a non-SOF S. pyogenes polypeptide.

30. The method of claim 27 wherein said fusion protein comprises one or more common immunogenic S. pyogenes SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

31. The method of claim 27 wherein said fusion protein comprises an immunogenic portion of a non-SOF-based polypeptide selected from the group consisting of S. pyogenes M protein, R28 protein, SPA, C5a peptidase, SFB1 (also know as protein F1), and FBP54.

32. A method for eliciting an in vivo antibody response against S. pyogenes in a mammal, said method comprising the step of administering to said mammal a cocktail comprising two or more immunogenic portions of a two or more S. pyogenes serum opacity factor polypeptides wherein said S. pyogenes serum opacity factor is selected from the group consisting of S. pyogenes SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

33. The method of claim 32 wherein one or more of said serum opacity factor polypeptides comprises a common immunogenic SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

34. The method of claim 32 wherein said cocktail comprises one or more immunogenic portion of an S. pyogenes serum opacity factor polypeptide and one or more immunogenic portion of a non-SOF S. pyogenes polypeptide.

35. The method of claim 32 wherein said cocktail comprises one or more common immunogenic S. pyogenes SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).

36. The method of claim 32 wherein said non-SOF-based polypeptide is selected from the group consisting of S. pyogenes M protein, R28 protein, SPA, C5a peptidase, SFB1 (also know as protein F1), and FBP54.

37. A method of treating an S. pyogenes infection in a mammal, said method comprising the step of administering to said mammal an antibody that specifically binds to an S. pyogenes serum opacity factor wherein said antibody is capable of facilitating opsonization of said S. pyogenes.

38. The method of claim 37 wherein said S. pyogenes serum opacity factor is selected from the group consisting of S. pyogenes SOF 2 (SEQ ID NO: 1), 4 (SEQ ID NO: 3), 8 (SEQ ID NO: 30), 9 (SEQ ID NO: 31), 11 (SEQ ID NO: 32), 13 (SEQ ID NO: 33), 15, 22 (SEQ ID NO: 34), 25 (SEQ ID NO: 35), 27 (SEQ ID NO: 36), 28 (SEQ ID NO: 5), 44 (SEQ ID NO: 37), 48 (SEQ ID NO: 38), 49 (SEQ ID NO: 39), 58 (SEQ ID NO: 40), 59 (SEQ ID NO: 41), 60 (SEQ ID NO: 42), 61 (SEQ ID NO: 43), 62 (SEQ ID NO: 44), 63 (SEQ ID NO: 45), 64, 66 (SEQ ID NO: 46), 68 (SEQ ID NO: 47), 73 (SEQ ID NO: 48), 75 (SEQ ID NO: 49), 76 (SEQ ID NO: 50), 77 (SEQ ID NO: 51), 78 (SEQ ID NO: 52), 79 (SEQ ID NO: 53), 81 (SEQ ID NO: 54), 87 (SEQ ID NO: 55), 103, 104, 106, 107, 109, 110, 112, 113, 114, 117, 118, and 124.

39. The method of claim 37 wherein said serum opacity factor is selected from the group consisting of SOF2 (SEQ ID NO: 1), SOF4 (SEQ ID NO: 3), and SOF28 (SEQ ID NO: 5).

40. The method of claim 37 wherein said serum opacity factor polypeptides comprises a common immunogenic SOF epitopes selected from the group consisting of ETEPQTMDVEQYTVDKENS (SEQ ID NO: 15), DIFDVKREVKTNGDGTLDVLT (SEQ ID NO: 16), PKQIDEGADVMALLDVSQKM (SEQ ID NO: 17), FDKAKEQIKKLVTTLT (SEQ ID NO: 18), YNRRNSVRLMTFYR (SEQ ID NO: 19), WGDVLQGAIHKAREIFNKEK (SEQ ID NO: 20), RQHIVLFSQGESTFSYDIK (SEQ ID NO: 21), TTSNPLFPWLPIFNHT (SEQ ID NO: 22), FDYSKRVGEGYYYHSFSDR (SEQ ID NO: 23), ERNEKFDNYLKEMSEGGK (SEQ ID NO: 24), DVDKADKFKDTLTEL (SEQ ID NO: 25), TKESLTWTISKD (SEQ ID NO: 26), and SLTLKYKLKVNKDKL (SEQ ID NO: 27).
